(19) United States
(12) Patent Application Publication
(10) Pub. No.: US 2026/0311873 A1
(43) Pub. Date: Oct. 8, 2026

(54) COMPUTE SYSTEM WITH IMAGE DIAGNOSTIC MECHANISM AND METHOD OF OPERATION THEREOF

(71) Applicant: BelleTorus Corporation, Cambridge, MA (US)

(72) Inventors: Thi Thu Hang Nguyen, Toulouse (FR); Folkert Blok, Toulouse (FR); Van Dong Tran, Toulouse (FR); Quang Do Duy, Hanoi (VN); Hoàng Lê, Hanoi (VN); Duc Thang NGUYEN, Toulouse (FR); Mariia Lebedeva, Toulouse (FR); Tien Dung Nguyen, Toulouse (FR); Jamy van den Brink, Wijk bij Duurstede (NL); Joannes Mathaeus Grevelink, Park City, UT (US)

(21) Appl. No.: 19/237,803

(22) Filed: Jun. 13, 2025

Related U.S. Application Data
(60) Provisional application No. 63/784,471, filed on Apr. 7, 2025.

(51) Int. Cl. G16H 50/20; G06T 7/00; G06V 10/25; G16H 10/20

(52) U.S. Cl. CPC G16H 50/20; G06T 7/0012; G06V 10/25; G16H 10/20; G06T 2207/30088; G06T 2207/30096

(57) ABSTRACT

A method of operation of a compute system includes: generating a skin region based on a patient image; identifying a region of interest from the skin region; generating a lesion type based on the region of interest; and generating a structural language output based on a category related to the lesion type for displaying on a device to assist in diagnosis.

### CROSS REFERENCE TO RELATED APPLICATION

This application claims the benefit of U.S. Provisional patent application Ser. No. 63/784,471 filed Apr. 7, 2025, and the subject matter thereof is incorporated herein by reference thereto.

### TECHNICAL FIELD

An embodiment of the present invention relates generally to a compute system, and more particularly to a system with an imaging diagnostic mechanism.

### BACKGROUND

The process of analyzing images with skin ailments in dermatological application is inherently complex, and providing visual and/or textual description from an artificial intelligence (AI) based system as an explainable AI to be understood by dermatologist and other clinical professionals is even more challenging.

Thus, a need still remains for a compute system with an AI based dermatological application for skin ailment to provide AI based approach that is explainable in text and/or images in meaningful terminology understandable by dermatologist and other clinical professions based on the original image. In view of the ever-increasing commercial competitive pressures, along with growing healthcare needs, healthcare expectations, and the diminishing opportunities for meaningful product differentiation in the marketplace, it is increasingly critical that answers be found to these problems. Additionally, the need to reduce costs, improve efficiencies and performance, and meet competitive pressures adds an even greater urgency to the critical necessity for finding answers to these problems.

Solutions to these problems have been long sought but prior developments have not taught or suggested any solutions and, thus, solutions to these problems have long eluded those skilled in the art.

### DISCLOSURE OF THE INVENTION

An embodiment of the present invention provides a method of operation of a compute system including: generating a skin region based on a patient image; identifying a region of interest from the skin region; generating a lesion type based on the region of interest; and generating a structural language output based on a category related to the lesion type for displaying on a device to assist in diagnosis.

An embodiment of the present invention provides a compute system, including: a control circuit, including a processor, configured to: generate a skin region based on a patient image; identify a region of interest from the skin region; generate a lesion type based on the region of interest; and generate a structural language output based on a category related to the lesion type for displaying on a device to assist in diagnosis.

An embodiment of the present invention provides a non-transitory computer-readable medium storing an instruction that, when executed by a control circuit of a compute system, causes the control circuit to perform functions including: generating a skin region based on a patient image; identifying a region of interest from the skin region; generating a lesion type based on the region of interest; and generating a structural language output based on a category related to the lesion type for displaying on a device to assist in diagnosis.

Certain embodiments of the invention have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an example of a system architecture diagram of a compute system with an image diagnostic mechanism in an embodiment of the present invention.

FIG. 2 is a first example of a user interface for the image diagnostic mechanism of FIG. 1.

FIG. 3 is a second example of the user interface displaying an inference generated by the image diagnostic mechanism of FIG. 1.

FIG. 4 is a third example of the user interface displaying an inference generated by the image diagnostic mechanism of FIG. 1 for a body part without an identified analysis region.

FIG. 5 is shown a fourth example of the user interface displaying an inference generated by the image diagnostic mechanism of FIG. 1 for the body part with identified analysis regions.

FIG. 6 is a fifth example of the user interface displaying an inference generated by the image diagnostic mechanism of FIG. 1 for the body part without an identified analysis region.

FIG. 7 is a flow overview of the image diagnostic mechanism of FIG. 1 along with an example output of a provoke AI module (table) and a description formed by a language model.

FIG. 8 is an example of a processing flow of the skin segmentation module of FIG. 7 in an embodiment.

FIG. 9 is an example for performance graphs of the skin segmentation module of FIG. 7 in an embodiment.

FIG. 10 is an example of a task of the body part segmentation module of FIG. 7 in an embodiment.

FIG. 11 are examples of lesion classes detected by the lesion detection module of FIG. 7 in an embodiment.

FIG. 12 is an example of an architecture of the lesion detection module of FIG. 7 in an embodiment.

FIG. 13 are examples of training performance graphs of the lesion detection module of FIG. 7 in an embodiment.

FIG. 14 are examples of validation performance graphs of the lesion detection module of FIG. 7 in an embodiment.

FIG. 15 are examples of performance graphs of metrics during training of the lesion detection module of FIG. 7 in an embodiment.

FIG. 16 is an exemplary block diagram of the compute system in an embodiment.

FIG. 17 is a flow chart of a method of operation of a compute system in an embodiment of the present invention.

### BEST MODE FOR CARRYING OUT THE INVENTION

Description of various embodiments of the present invention is described with an example of a dermatological application for skin ailment, and as a specific example of explainable AI (XAI) to describe the skin ailment. Artificial intelligence (AI), as a specific example deep learning, has been successful at detecting skin cancer from a lesion image, but its practical adoption is limited by the lack of explanation behind its decisions or relate the inference in a language meaningful to a dermatologist or medical or clinical professional.

The following embodiments are described in sufficient detail to enable those skilled in the art to make and use the invention. It is to be understood that other embodiments would be evident based on the present disclosure, and that system, process, or mechanical changes may be made without departing from the scope of an embodiment of the present invention.

In the following description, numerous specific details are given to provide a thorough understanding of the invention. However, it will be apparent that the invention may be practiced without these specific details. In order to avoid obscuring an embodiment of the present invention, some well-known circuits, system configurations, and process steps are not disclosed in detail.

The drawings showing embodiments of the system are semi-diagrammatic, and not to scale and, particularly, some of the dimensions are for the clarity of presentation and are shown exaggerated in the drawing figures. Similarly, although the views in the drawings for ease of description generally show similar orientations, this depiction in the figures is arbitrary for the most part. Generally, the invention can be operated in any orientation. The embodiments of various components as a matter of descriptive convenience and are not intended to have any other significance or provide limitations for an embodiment of the present invention.

The term “module” or “unit” or “circuit” referred to herein can include or be implemented as or include software running on specialized hardware, hardware, or a combination thereof in the present invention in accordance with the context in which the term is used. For example, the software can be machine code, firmware, embedded code, and application software. The software can also include a function, a call to a function, a code block, or a combination thereof.

Also, for example, the hardware can be gates, circuitry, processor, computer, integrated circuit, integrated circuit cores, memory devices, a pressure sensor, an inertial sensor, a microelectromechanical system (MEMS), passive devices, physical non-transitory memory medium including instructions for performing the software function, a portion therein, or a combination thereof to control one or more of the hardware units or circuits. Further, if a “module” or “unit” or a “circuit” is written in the claims section below, the “unit” or the “circuit” is deemed to include hardware circuitry for the purposes and the scope of the claims.

The module, units, or circuits in the following description of the embodiments can be coupled or attached to one another as described or as shown. The coupling or attachment can be direct or indirect without or with intervening items between coupled or attached modules or units or circuits. The coupling or attachment can be by physical contact or by communication between modules or units or circuits, such as wireless communication.

The word “module” or “model” can be used interchangeable depending on the context it is described or used in the written description. The “model” can represent one or more artificial intelligence models, machine learning models, or a combination thereof.

It is also understood that the nouns or elements in the embodiments can be described as a singular instance. It is understood that the usage of singular is not limited to singular but the singular usage can be applicable to multiple instances for any particular noun or element in the application. The numerous instances can be the same or similar or can be different.

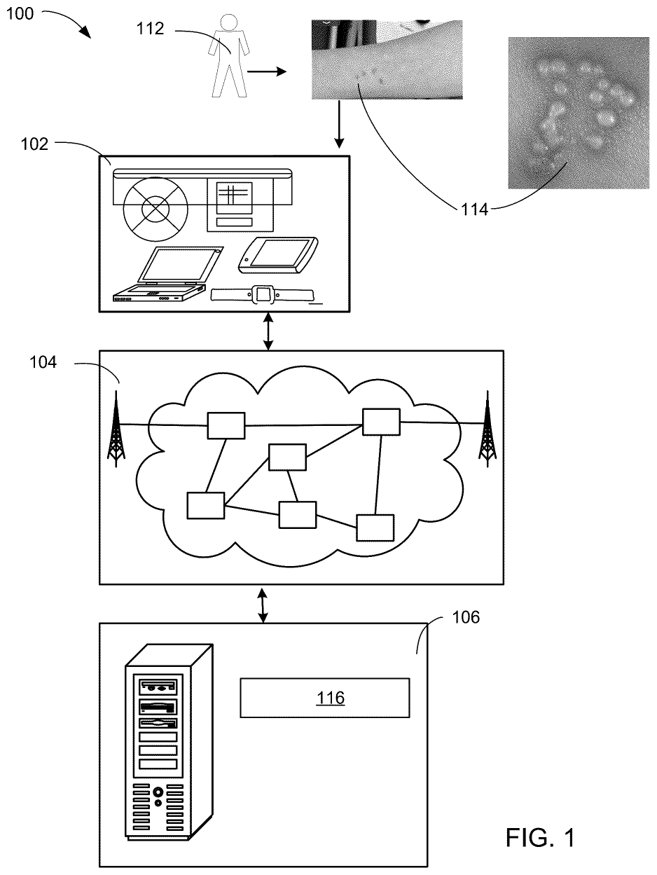

Referring now to FIG. 1, therein is shown an example of a system architecture diagram of a compute system 100 with an image diagnostic mechanism 116 in an embodiment of the present invention. One or more embodiments of the compute system 100 provide terminology-based approach, for example in the field of dermatology or the medical field, to improve AI interpretability, interactivity, accuracy, or a combination thereof in assessing skin disease severity, such as one or more lesions. By generating terminology-based interpretable output in the respective field for what is being examined or analyzed by the compute system 100, the doctors, the clinicians, or staff members of the clinicians as well as the ecosystem for that field can better interact with and utilize the output of the compute system 100.

For brevity, the application of the embodiment described will be for PROVOKE (in Dutch) or acronym used as a model to describe skin abnormalities (efflorescences). The acronym PROVOKE in Dutch stands for P (plaats), R (rangschikking), O (omvang), V (vorm), O (omtrek), K (kleur), and E (efflorescentie). In English, these Dutch words can refer to place, arrangement, circumference (e.g., lenticular to nummular (1-2 cm) large), shape, circumference (e.g., sharply delineated), color, and efflorescence. Efflorescences (Latin for ‘blooms’) are defined as changes in skin that can be typical of a particular skin disease, and that can affect the skin disease. A skin disease can be made up of one or more efflorescences, for example, one or more papules or plaques.

The acronym for PROVOKE is expanded as an example as followed: P: on trunk and proximal part extremities, R: symmetrically distributed, oval lesions with longitudinal axis according to the cleavage lines of the skin (pine tree configuration), O: lenticular to nummular (1-2 cm) large, V: oval-shaped pink-red spots with pityriasiform scaling, and the older ones show central bleaching and a fine flaky border within the red edge (medallion), where “the older ones” can refer to the lesions (e.g., oval pink-red spots) that have been present on the skin for a longer time, i.e., the more mature or aged-in lesions compared with freshly-appeared ones, O: sharply delineated, K: pink red/pale center, and E: erythematosquamous plaques, which can be very rarely vesicular, vesiculobullous. One or more embodiments can be implemented for other fields, terminologies, ailments, and applications but for brevity and clarity of describing the invention, PROVOKE is a description vehicle and not intended to be limiting to PROVOKE or the application to dermatology or medical fields.

The compute system 100 can include a first device 102, such as a client or a server, connected to a second device 106, such as a client or server. The first device 102 can communicate with the second device 106 through a network 104, such as a wireless or wired network.

For example, the first device 102 can be of any of a variety of computing devices, such as a smart phone, a tablet, a cellular phone, personal digital assistant, a notebook computer, a wearable device, internet of things (IoT) device, or other multi-functional device. Also, for example, the first device 102 can be included in a device or a sub-system.

The first device 102 can couple, either directly or indirectly, to the network 104 to communicate with the second device 106 or can be a stand-alone device. The first device 102 can further be separated from or incorporated with a smart phone, a tablet computer, a laptop computer, a scanner, or other personal electronic devices.

For illustrative purposes, the compute system 100 is described with the first device 102 as a mobile device, although it is understood that the first device 102 can be different types of devices. For example, the first device 102 can also be a non-mobile computing device, such as a server, a server farm, cloud computing, or a desktop computer.

The second device 106 can be any of a variety of centralized or decentralized computing devices. For example, the second device 106 can be a computer, grid computing resources, a virtualized computer resource, cloud computing resource, routers, switches, peer-to-peer distributed computing devices, or a combination thereof.

The second device 106 can be centralized in a single room, distributed across different rooms, distributed across different geographical locations, embedded within a telecommunications network. The second device 106 can be coupled with the network 104 to communicate with the first device 102. The second device 106 can also be a client type device as described for the first device 102.

For illustrative purposes, the compute system 100 is described with the second device 106 as a non-mobile computing device, although it is understood that the second device 106 can be different types of computing devices. For example, the second device 106 can also be a mobile computing device, such as notebook computer, another client device, a wearable device, or a different type of client device.

Also, for illustrative purposes, the compute system 100 is described with the second device 106 as a computing device, although it is understood that the second device 106 can be different types of devices. Also, for illustrative purposes, the compute system 100 is shown with the second device 106 and the first device 102 as endpoints of the network 104, although it is understood that the compute system 100 can include a different partition between the first device 102, the second device 106, and the network 104. For example, the first device 102, the second device 106, or a combination thereof can also function as part of the network 104.

The network 104 can span and represent a variety of networks. For example, the network 104 can include wireless communication, wired communication, optical, ultrasonic, or the combination thereof. Satellite communication, cellular communication, Bluetooth, Infrared Data Association standard (IrDA), wireless fidelity (WiFi), and worldwide interoperability for microwave access (WiMAX) are examples of wireless communication that can be included in the communication path. Ethernet, digital subscriber line (DSL), fiber to the home (FTTH), and plain old telephone service (POTS) are examples of wired communication that can be included in the network 104. Further, the network 104 can traverse a number of network topologies and distances. For example, the network 104 can include direct connection, personal area network (PAN), local area network (LAN), metropolitan area network (MAN), wide area network (WAN), or a combination thereof.

For example, the compute system 100 can provide the functions for the patients 112 or other users, such as doctors, clinicians, or other healthcare professionals, working with the patients 112 with the first device 102, the second device 106, distributed between these two devices, or a combination thereof. Also as examples, the compute system 100 can provide a mobile applications for the patients, the clinicians, or a combination thereof. Further as an example, the compute system 100 can provide the functions via a web-browser based applications or a software to be executed on the first device 102, the second device 106, distributed between these two devices, or a combination thereof.

In one embodiment as an example, patient images 114 are taken and uploaded by the patient 112 or for the patient 112 and reviewed by the clinician. In this embodiment, a patient 112 launches the image diagnostic mechanism 116 via the mobile application and logs into the patient's account. The patient 112 or the user of the mobile application can be prompted to upload or take images as the patient images 114. The compute system 100 can guide a patient 112 or a user of the mobile application on photo guidelines for the patient images 114 and accepts or rejects the patient images 114 for retake based on a pre-specified criteria, e.g., distance, quality, blur, or a combination thereof. The compute system 100 can also provide guides for a patient 112 or another user on capturing videos as opposed to still photos. Also for example, the patient images 114 can be selected from the video.

Once the patient images 114 are successfully uploaded, the compute system 100 can send or load the patient images 114 to the image diagnostic mechanism 116 for analysis. The image diagnostic mechanism 116 will be described later. For brevity and clarity and as an example, the image diagnostic mechanism 116 is shown as being executed in the second device 106 although it is understood that portions of the image diagnostic mechanism 116 can operate on the first device 102, such as the mobile application or the web-browser based application, can operate completely on the first device 102, or a combination thereof. The image diagnostic mechanism 116 can be implemented in software running on specialized hardware, full hardware, or a combination thereof.

Based on analysis results, the compute system 100 can display information to the patient 112 including a recommendation based on the patient images 114, uploaded, for the patient 112 to schedule a visit with a primary care physician or with a specialist.

Continuing the example, the compute system 100 can provide a function that allows the clinician to access the patient images 114 uploaded by the patient 112 or for the patient 112 and the image diagnostic mechanism 116, such as with the web-based dashboard. The compute system 100 allows the clinician to make edits to annotations determined by the image diagnostic mechanism 116 and the scores (if necessary) and save the results. The clinician can utilize the image diagnostic mechanism 116 to make the diagnostic decision and suggest necessary treatment steps (if applicable).

For example, executing the image-diagnostic mechanism 116 on the first device 102 at the edge can have faster inference time. For another example, the first device 102 can interface with the second device 106 via the network 104 to use more powerful processing capability of the second device 106 to execute the image-diagnostic mechanism 116 compared to the processing capability of the first device 102 to provide faster inference time. In these examples, the faster inference time provided by the first device 102, the second device 106, or a combination thereof applies to execution of quality check, skin segmentation, lesion detection, body part segmentation, differential diagnosis, provoke AI, language model, provoke database, clinician input, structured table, description and questions, questions panel, or a combination thereof.

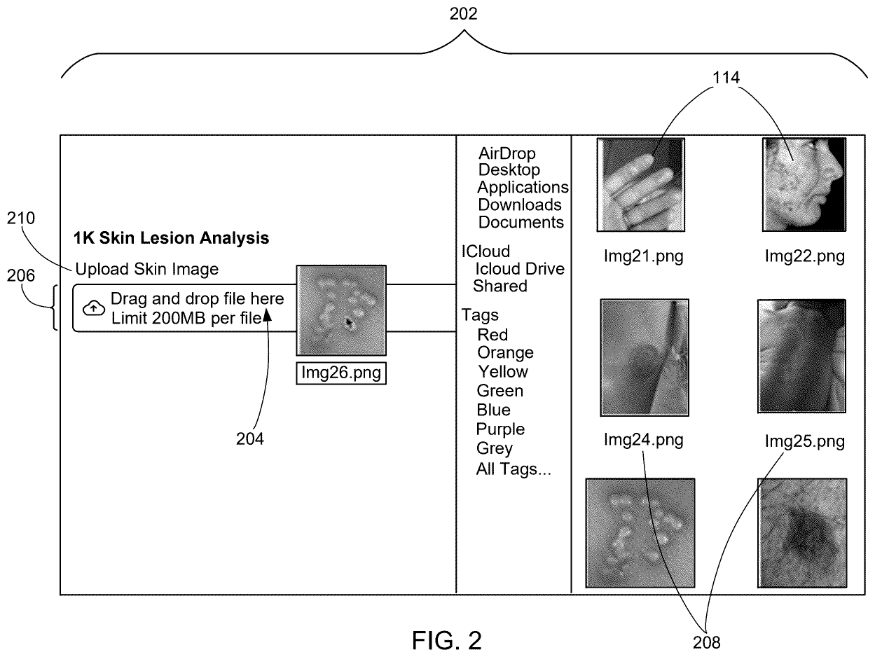

Referring now to FIG. 2, therein is shown a first example of a user interface 202 for the image diagnostic mechanism 116 of FIG. 1. By way of an example, the user interface 202 is an interactive presentation layer that enables information exchange between end users and the compute system 100. In this example embodiment, the patient images 114 are input to the image diagnostic mechanism 116 through the user interface 202.

For example, the user interface 202 can provide functionality for uploading patient images 114 through an intuitive drag-and-drop action 204. By way of an example, the drag-and-drop action 204 is a pointer-driven gesture that transfers data objects from a source location to a target location within the presentation layer.

For example, the user interface 202 can include a defined image upload region 206 prompting users, such as the patients 112 of FIG. 1 or the clinicians, to drag patient image files 208 of the patient images 114 directly from a local file storage into the designated area. By way of an example, the defined image upload region 206 is a bounded screen area designated to receive files introduced via placement or selection operations. By way of an example, the patient image files 208 are digital file instances that include visual data of a subject for diagnostic analysis.

Additionally also an example of a further embodiment, the user interface 202 can include an interactive upload button 210 for alternatively selecting the patient image files 208 from a local directory, providing flexibility in how the patient images 114 are introduced into the compute system 100 of FIG. 1. By way of an example, the upload button 210 is a selectable control element that initiates file-selection and transfer operations. Visual cues within the image upload region 206 can guide the positioning of the patient image files 208, reducing potential user errors during image submission. One or more embodiments can support both single-image and multi-image uploads, accommodating varying diagnostic needs.

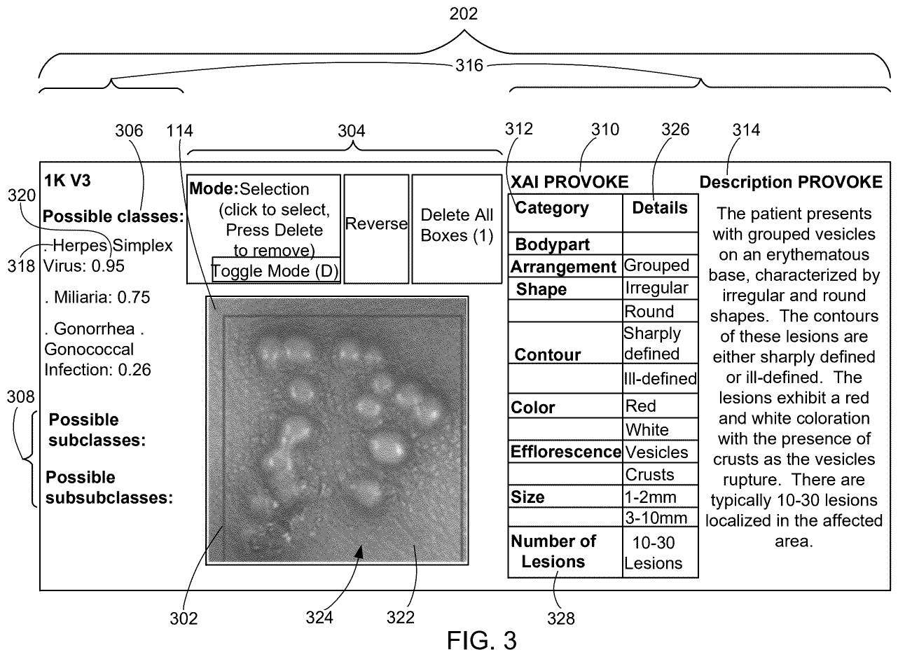

Referring now to FIG. 3, therein is shown a second example of the user interface 202 displaying an inference generated by the image diagnostic mechanism 116 of FIG. 1. For example, the inference visually encloses the analyzed skin area with a bounding box 302 (e.g., also referred to as a red box or skin-region-of-interest). By way of an example, the bounding box 302 is a rectangular frame that delineates a targeted area within an image for analysis.

For example, the bounding box 302 is colored red to distinguish it from any additional bounding boxes that can be also generated. For example, the image diagnostic mechanism 116 automatically draws the red bounding box 302 around or at the highest-priority region of interest, while a clinician can create supplementary blue boxes by switching the annotation-control panel 304 to Drawing mode and dragging further rectangles, thereby allowing automatic and interactive region selection within the same workflow.

For example, an annotation-control panel 304 accompanies the bounding box 302 and exposes tools such as “Toggle Mode,” “Reverse,” and “Delete All Boxes” for manipulating the annotation. By way of an example, the annotation-control panel 304 is a collection of interactive controls that governs annotation operations within the presentation layer.

As an example, diagnostic feedback is presented visually on the patient images 114 and textually on the left side of the user interface 202, outlining the types of skin ailments identified and associated likelihood metrics. As an example, the left side includes possible disease classes 306 such as Herpes Simplex Virus 0.95, Miliaria 0.75, and Gonorrhea 0.026. By way of an example, the possible disease classes 306 are an ordered list that presents candidate disorders together with associated probability scores.

For example, each possible disease class 306 includes a disease 318, which is a textual identifier that specifies a candidate medical condition associated with an analyzed region, and a likelihood score 320, which is a numerical value that represents the confidence level the compute system 100 assigns to the corresponding candidate medical condition. As an example, the likelihood score 320 can include a range from 0.00 to 1.00, with an increasing value indicates that there is an increase likelihood that the candidate medical condition is present, where 0.00 indicates that a likelihood of a candidate medical condition is 0% present and 1.00 indicates that a likelihood of a candidate medical condition is 100% present. As an illustrative specific example, a pair of the disease 318 and the likelihood score 320 can be Herpes Simplex Virus and 0.95, Miliaria and 0.75, and Gonorrhea and 0.026, as examples.

As an example, a subclass list 308 (e.g., also referred to as possible-subclasses and possible-subclasses lists) provides successively deeper taxonomic levels beneath the possible disease classes 306. By way of an example, the subclass list 308 is an ordered group that enumerates subordinate diagnostic categories beneath a primary class. For example, each subclass list 308 includes the disease 318 and the likelihood score 320.

For example, to the right of the patient images 114, the user interface 202 presents a structured table 310 (e.g., also referred to as an XAI PROVOKE table or structured diagnostic table) whose rows each bear a category 312. By way of an example, the category 312 is a label that designates a descriptive field such as body part, arrangement, shape, contour, color, efflorescence, size, or number of lesions. By way of an example, the structured table 310 is a grid-based arrangement that lists categorical descriptors alongside their corresponding values for interpretability. For example, specific diagnostic text structured according to the PROVOKE dermatological standard is shown, detailing Primary lesion, Secondary lesion, Shape, Distribution, Arrangement, Color, and Border. For example, specific annotations visible in FIG. 2 include “Primary lesion: papule,” “Secondary lesion: crust,” and “Arrangement: grouped,” each accompanied by a likelihood percentage to provide clarity on diagnostic confidence.

As an example, above the structured table 310, the user interface 202 renders a diagnostic text block 314 (e.g., also referred to as Description PROVOKE) that narratively summarizes the analytical observations. By way of an example, the diagnostic text block 314 is a narrative segment that summarizes analytical observations using natural language. As an example, under the heading labeled “Description PROVOKE,” the inference text is generated by the image diagnostic mechanism 116 and is structured according to the PROVOKE dermatological standard which includes Primary lesion, Secondary lesion, Shape, Distribution, Arrangement, Color, Border, and additional dermatological descriptors. As an example, each of these structured categories includes precise descriptive terms automatically generated by the image diagnostic mechanism 116 reflecting specific observations made by the AI during image analysis.

For example, the bounding box 302 guides the user, such as the patient 112, the clinician, or a combination thereof, visually to the exact location corresponding to the textual diagnostic inferences and clearly indicates the area of concern on the patient's arm, facilitating easy correlation between textual descriptions and physical symptoms visually presented in the image. For example, the red box can be generated by the image diagnostic mechanism 116 as detection of a skin region 322 of interest or can be included in the patient images 114 or inserted by a user via the annotation-control panel 304.

By way of an example, the skin region 322 is a contiguous portion of patient tissue selected for analysis. As an example, the skin region 322 is the portion of the patient image 114 that lies inside the bounding box 302. For example, the rectangle representing the bounding box 302 encloses an area of skin-free of clothing and background—that the image diagnostic mechanism 116 selects for detailed analysis after skin segmentation is confirmed that the skin region 322 contains only skin pixels. In this example, within the skin region 322, the lesions appear as oval, pink-red macules and papules with light scaling, matching the PROVOKE descriptors later listed in the structured table 310. For example, everything outside the skin region 322 includes shirt fabric and background and is therefore excluded from the skin region 322 used for diagnosis.

For example, a region of interest 324 is a rectangular zone outlined by the bounding box 302 that appears atop the patient image 114. By way of an example, the region of interest 324 is a bounded portion of the skin region 322 selected for lesion analysis. As an example, the region of interest 324 lies wholly inside the skin region 322.

As an example, the structured, detailed breakdown of the diagnostic text block 314 together with the category 312 rows of the structured table 310 and the probability scores in the possible disease classes 306 makes the inference text a form of explainable AI (XAI) because it transparently outlines how the AI arrived at its diagnostic conclusions. As an example, the AI-generated descriptions clearly state the types of lesions identified such as macules, papules, and plaques, the specific shape such as round, oval, or irregular, color variations, and the distribution pattern such as localized, symmetrical, or diffuse. As an example, by presenting detailed reasoning and clearly linked observations that correlate directly with visual annotations in the patient images 114, the compute system 100 of FIG. 1 enhances the interpretability of AI-generated outputs, thereby allowing clinicians, such as medical practitioners or dermatologists, to validate the AI's diagnostic process and facilitating trust, understanding, clinical acceptance, and informed decision-making.

As an example, the image diagnostic mechanism 116 aggregates the possible disease classes 306, the structured table 310, and the diagnostic text block 314 into a structural language output 316. By way of an example, the structural language output 316 is a compound presentation that aggregates the possible disease classes 306, the structured table 310, and the diagnostic text block 314 into a single interpretable report.

It has been unexpectedly discovered that structural language output 316, which collocates the possible disease classes 306, the structured table 310, and the diagnostic text block 314, enables clinicians to validate model reasoning at a glance, thereby greatly reducing the time needed to reach a confident diagnosis while maintaining transparency and trust.

For example, the structured table 310 includes a feature 326 that appears as the specific value(s) entered for each category 312. By way of an example, the feature 326 is an attribute value that populates a descriptor field. For example, each textual or numeric value of the feature 326 is calculated from pixel data inside the region of interest 324 (bounded by the bounding box 302) and then written into the corresponding row of the structured table 310.

For example, the category 312 includes a number of lesions 328. By way of an example, the number of lesions 328 is a category, whose value quantifies discrete dermatological findings within an analysis zone. For example, a value of the number of lesions 328 is an integer recorded in the row of the number of lesions 328 of the structured table 310 for the region of interest 324 enclosed by the bounding box 302. As a specific example, the lesion detection module 706 tallies every discrete papule or macule visible inside the bounding box 302, while ignoring background pixels filtered out by the skin segmentation, and transmits that count to the provoke AI module 712, which writes the value into the structured table 310 and also passes the count to the language model module 714.

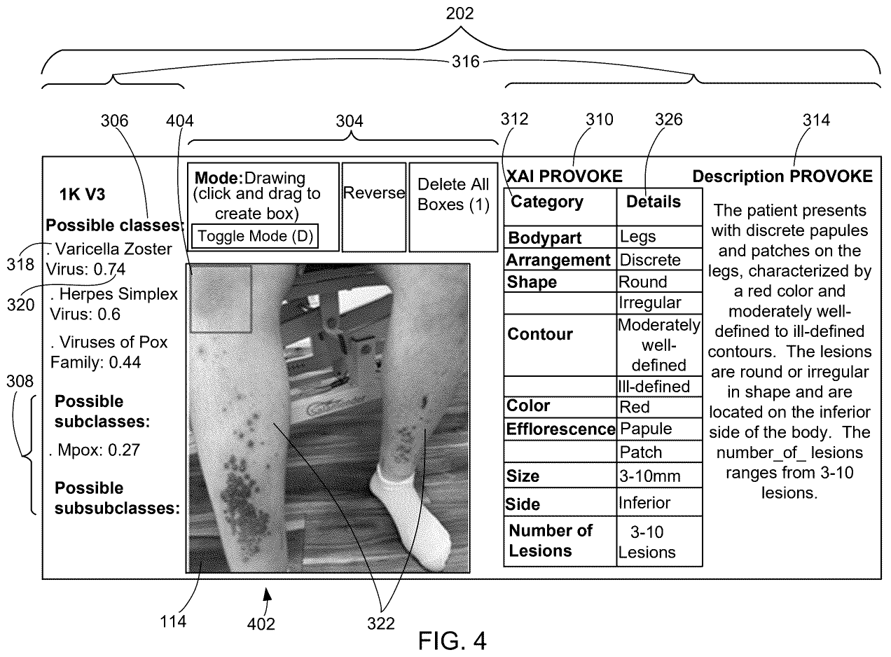

Referring now to FIG. 4, therein is shown a third example of the user interface 202 displaying an inference generated by the image diagnostic mechanism 116 of FIG. 1 for a body part 402 without an identified analysis region. For example, the inference is visually provided as diagnostic feedback on the patient image previously uploaded, similar to FIG. 3. As an example, this depiction also generates diagnostic feedback without a defined region of interest or visual annotations, such as bounding boxes 302 of FIG. 3, on the patient images 114.

For example, the middle portion of the user interface 202 includes the patient image 114 depicting a broader area, including a body region along with surrounding non-body areas, such as background elements, clothing, flooring, or other irrelevant image portions related to the skin region 322, the skin ailment, or a combination thereof. This is in contrast to FIG. 3, which specifically shows only a focused skin portion clearly marked and highlighted by a red bounding box 302 of FIG. 3.

For example, the image diagnostic mechanism 116, a clinician, or a combination thereof can create supplementary boxes 404 as regions of interest by switching the annotation-control panel 304 to Drawing mode and dragging rectangles, thereby allowing automatic and interactive region selection within the same workflow. By way of an example, the supplementary boxes 404 is a set of secondary region-of-interest frames that delineate additional areas within an image for analysis. As an illustrative example, a supplementary box 404 is shown as a blue box in the skin region 322 of the body part 402 (e.g., leg) in the patient image 114. As an example, the bounding box 302 of FIG. 3 is in red to mark the primary or first region of interest, whereas the supplementary boxes 404 are in blue to denote additional regions that merit examination after the primary region of interest (ROI).

For example, an annotation-control panel 304 situated above patient images 114 provides interactive buttons labelled “Mode: Drawing (click and drag to create box),” “Toggle Mode (D),” “Reverse,” and “Delete All Boxes (1),” thereby allowing creation, modification, and deletion of the bounding box 302 of FIG. 3.

As an example, a list of possible disease classes 306 appears on the left side of the user interface 202 and enumerates Varicella Zoster Virus 0.74, Herpes Simplex Virus 0.6, and Viruses of Pox Family 0.44.

For example, each possible disease class 306 includes a disease 318, which is a textual identifier that specifies a candidate medical condition associated with an analyzed region, and a likelihood score 320, which is a numerical value that represents the confidence level the compute system 100 assigns to the corresponding candidate medical condition. As an example, the likelihood score 320 can include a range from 0.00 to 1.00, with an increasing value indicates that there is an increase likelihood that the candidate medical condition is present, where 0.00 indicates that a likelihood of a candidate medical condition is 0% present and 1.00 indicates that a likelihood of a candidate medical condition is 100% present. As an illustrative specific example, a pair of the disease 318 and the likelihood score 320 can be Herpes Simplex Virus and 0.95, Miliaria and 0.75, and Gonorrhea and 0.026, as examples.

For example, a subclass list 308 directly beneath possible disease classes 306 supplies deeper taxonomic suggestions such as Mpox 0.27. For example, each subclass list 308 includes the disease 318 and the likelihood score 320. For illustrative specific example, the subclass list 308 includes the disease 318 of Mpox and the likelihood score 320 of 0.27.

As an example, a structured table 310 is displayed to the right of patient images 114, wherein each category 312—body part 402, arrangement, shape, contour, color, efflorescence, size, side, and number of lesions—is paired with respective values of the feature 326, such as legs, discrete, round/irregular, moderately well-defined/ill-defined, red, papule/patch, 3-10 mm, inferior, and 3-10 lesions. By way of an example, the body part 402 is an anatomical location descriptor that identifies where on the subject the lesions are situated.

As an example, a diagnostic text block 314 under the heading “Description PROVOKE” narratively summarizes the analytical observations. As an example, under the heading labeled “Description PROVOKE,” the inference text is generated by the image diagnostic mechanism 116 and is structured according to the PROVOKE dermatological standard which includes Primary lesion, Secondary lesion, Shape, Distribution, Arrangement, Color, Border, and additional dermatological descriptors. Like the embodiment in FIG. 3, the diagnostic text block 314 optionally generated by the image diagnostic mechanism 116 can describe potential skin ailments or dermatological conditions, along with metrics indicating likelihood, severity, or confidence levels of the diagnostic assessments. For example, the possible disease classes 306, the structured table 310, and the diagnostic text block 314 together compose the structural language output 316 that contextualizes an interpretable, multi-layered explanation.

For example, FIG. 4 demonstrates the flexibility of the image diagnostic mechanism 116 by providing meaningful diagnostic information even without explicitly defined or user-selected analysis regions, enabling diagnostic assessment of broader images containing multiple regions or background elements. As an example, in contrast, FIG. 3 explicitly provides visual indications such as the red box correlated directly to diagnostic text, thereby enhancing explainability through a more targeted analysis of specific skin areas. For example, these differences highlight alternative modes of operation of the image diagnostic mechanism, allowing diagnostic assessments either across precisely defined the skin region 322 or across broader image areas encompassing both body and non-body portions, accommodating various clinical workflows or user preferences.

It has been unexpectedly discovered that structural language output 316, produced from elements 306, 310, and 314, allows clinicians to validate model reasoning rapidly-even when broad images contain substantial background clutter-thereby accelerating diagnosis while preserving transparency and trust.

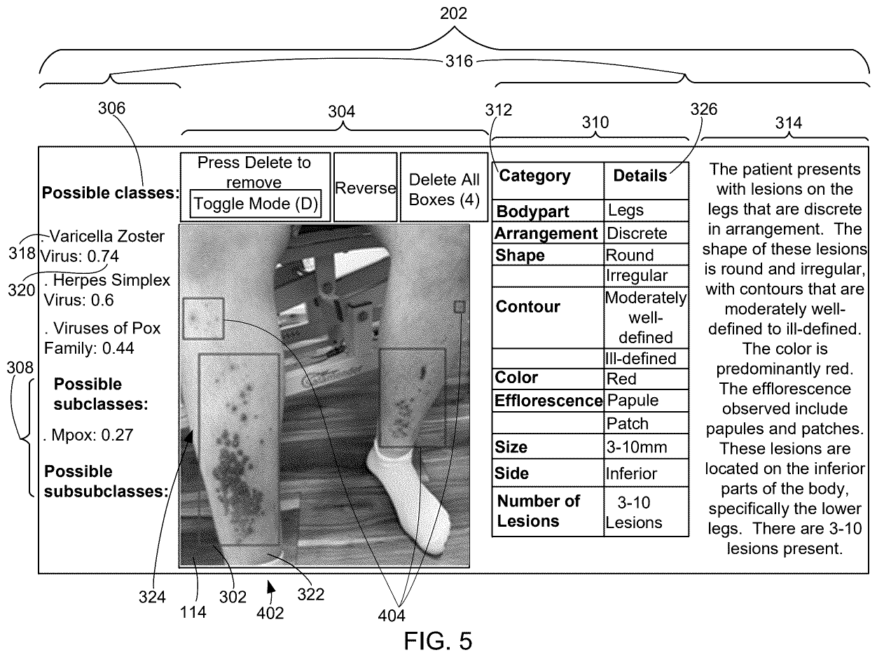

Referring now to FIG. 5, therein is shown a fourth example of the user interface 202 displaying an inference generated by the image diagnostic mechanism 116 of FIG. 1 for the body part 402 with identified analysis regions. For example, the inference is visually provided as diagnostic feedback on a patient image 114 previously uploaded, similar to FIG. 3 and FIG. 4. For example, the middle portion of the user interface 202 displays patient images 114 that show both legs of the subject against background elements such as a wooden floor, a portion of a bed, and a sock, as examples.

For example, a bounding box 302 (e.g., red box) and additional boxes (e.g. 3 blue boxes) highlight discrete analysis regions on the legs, and the annotation-control panel 304 displays the status “Delete All Boxes (4)”, indicating the total number of regions. For example, unlike FIG. 4, which shows broader, unmarked regions, FIG. 5 uses visual annotations to specifically indicate multiple areas or multiple of the region of interest 324, focusing diagnostic attention separately on each selected skin region 322 and thereby reducing ambiguity from background or irrelevant image portions. For example, the image diagnostic mechanism 116 can generate the bounding boxes 302 for the one or more skin regions 322 that can be inflicted with one or more skin ailments and can depict them with different colors, geometric shapes, line thicknesses, or dashed lines depending on differences in potential types of skin ailments.

For example, the possible disease classes 306 list enumerates the disease 318 and the likelihood score 320 pairs such as Varicella Zoster Virus 0.74, Herpes Simplex Virus 0.60, and Viruses of Pox Family 0.44. For example, a subclass list 308 immediately beneath includes Mpox 0.27 as a deeper differential.

For example, a structured table 310 displays each category 312 and the corresponding feature 326: body part 402 and legs, arrangement and discrete, shape and round/irregular, contour and moderately well-defined/ill-defined, color and red, efflorescence and papule/patch, size and 3-10 mm, side and inferior, and number of lesions and 3-10 lesions.

For example, a diagnostic text block 314 narratively summarizes the analytical observations using the information of the category 312 and the corresponding feature 326 in the structured table 310. For example, if the skin ailments differ between bounding boxes 302, the diagnostic text block 314 can clearly indicate distinct dermatological conditions or varying descriptors for each region. Alternatively, if multiple bounding boxes 302 share similar or identical skin ailments, the image diagnostic mechanism 116 can provide annotations or notations indicating the similarity or equivalence in diagnostic findings, avoiding redundancy and improving interpretability.

For example, the possible disease classes 306, the structured table 310, and the diagnostic text block 314 collectively form the structural language output 316 for each bounding box 302. For example, similar to FIG. 3 and optionally FIG. 4, the diagnostic text block 314 can also follow the structured dermatological PROVOKE format, correlating descriptors such as Primary lesion, Secondary lesion, Shape, Distribution, Arrangement, Color, Border, and additional dermatological attributes to each visually indicated analysis region independently.

For example, the bounding box 302 is in red color to distinguish the bounding box 302 from any additional supplementary boxes 404 that can be generated. For example, the image diagnostic mechanism 116 automatically generates the red bounding box 302 around or at the highest-priority region of interest (ROI), such as the region of interest 324, while a clinician can create supplementary boxes 404 by switching the annotation-control panel 304 to Drawing mode and dragging further rectangles, thereby allowing automatic and interactive region selection within the same workflow.

For example, the bounding box 302 is in red to mark the primary or first region of interest, whereas the supplementary boxes 404 are in blue to denote additional regions that merit examination after the primary ROI. Also as example, the bounding box 302 is drawn automatically around the highest-priority lesion cluster (or the first box a clinician draws), while each supplementary box 404 appears when the image-diagnostic mechanism 116 detects further clusters or when the clinician manually drags extra rectangles in “Drawing” mode. Further as an example, the user interface 202 can display every supplementary box 404 that links to a separate disease-probability list and PROVOKE descriptors. As an example, the annotation-control panel 304 tallies all boxes (e.g., “Delete All Boxes (4)”), letting the patient 112 of FIG. 1, the clinician, or a combination thereof to manage primary and supplementary ROIs within one workflow.

It has been unexpectedly discovered that presenting distinct colors, explicit box counts in the annotation-control panel 304, and detailed structural language output 316 for each bounding box 302 enables clinicians to compare multiple lesion clusters simultaneously while retaining high interpretability and diagnostic confidence.

For example, FIG. 5 illustrates the capability of the image diagnostic mechanism 116 to adaptively provide detailed, targeted, and visually correlated diagnostic information for multiple explicitly identified analysis regions, complementing the broader, non-annotated image diagnostic capabilities shown in FIG. 4 and aligning with the structured and visually correlated diagnostics depicted in FIG. 3, thereby collectively demonstrating the flexibility of the image diagnostic mechanism 116 across multiple clinical workflows and scenarios. 4 and aligning with the structured, visually correlated diagnostics depicted in FIG. 3.

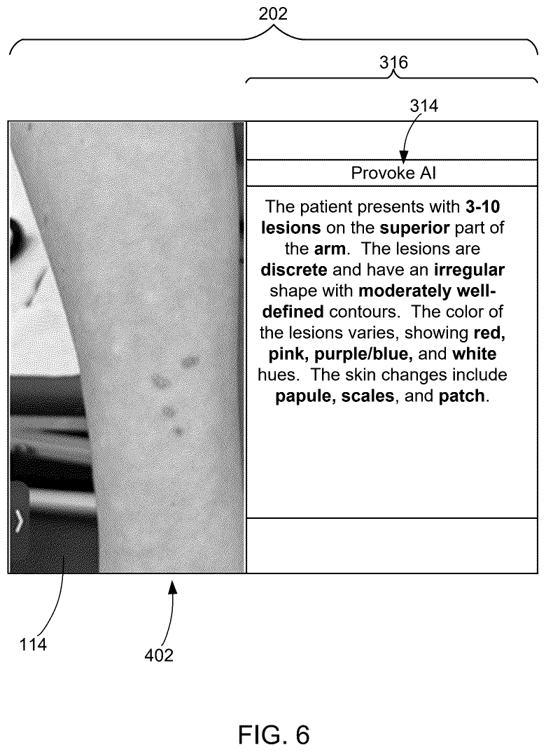

Referring now to FIG. 6, therein is shown a fifth example of the user interface 202 displaying an inference generated by the image diagnostic mechanism 116 of FIG. 1 for the body part 402 without an identified analysis region. For example, the inference is visually provided as diagnostic feedback on a patient image 114 previously uploaded, similar to FIGS. 3, 4, and 5, and FIG. 6 relates to the embodiment shown in FIG. 4 by similarly generating diagnostic feedback without predefined or visually annotated regions (e.g. without the bounding box 302), differing from FIGS. 3 and 5, which identify analysis regions through the bounding boxes 302.

As an example, the middle portion of the user interface 202 prominently displays patient images 114 depicting the body part 402 (e.g. arm) together with surrounding background elements, clothing, or other non-body portions, and no ROI rectangles, such as the bounding box 302, appear in FIG. 6, thereby contrasting with FIGS. 3 and 5, which utilize the bounding box 302 annotations.

For example, diagnostic inferences generated by the image diagnostic mechanism 116 are displayed adjacent to the patient images 114, similarly structured to FIGS. 3-5, and the diagnostic text block 314 describes potential dermatological conditions detected across the displayed body region and can include clinical observations, descriptive dermatological terms, and associated metrics indicating likelihood, severity, or confidence levels.

As an example, the diagnostic text block 314 states verbatim: “The patient presents with 3-10 lesions on the superior part of the arm. The lesions are discrete and have an irregular shape with moderately well-defined contours. The color of the lesions varies, showing red, pink, purple/blue, and white hues. The skin changes include papule, scales, and patch.” As an example, in certain embodiments, this diagnostic inference text also follows the structured dermatological PROVOKE format, providing clear clinical descriptors such as Primary lesion, Secondary lesion, Shape, Distribution, Arrangement, Color, Border, and additional attributes, consistent with the structured outputs previously described in FIGS. 3-5.

For example, FIG. 6 illustrates the adaptability and flexibility of the image diagnostic mechanism 116, effectively supporting diagnostic assessments of various body regions without annotated areas, much like the embodiment illustrated in FIG. 4, while FIG. 5 provides targeted and visually annotated diagnostic feedback through clearly defined regions, emphasizing precise correlation between visual annotations and diagnostic text. As an example, collectively FIGS. 3 through 6 illustrate multiple modes of operation for the image diagnostic mechanism 116, accommodating both annotated (FIGS. 3 and 5) and non-annotated (FIGS. 4 and 6) approaches, thereby meeting diverse clinical needs and workflow preferences.

It has been unexpectedly discovered that providing color-coded descriptive text without bounding-box overlays allows rapid triage of full-arm photographs when precise ROI selection is impractical, because the user interface 202, the patient image 114, the structural language output 316, or a combination thereof delivers clinically rich information while eliminating the additional interaction steps required for drawing the bounding boxes 302.

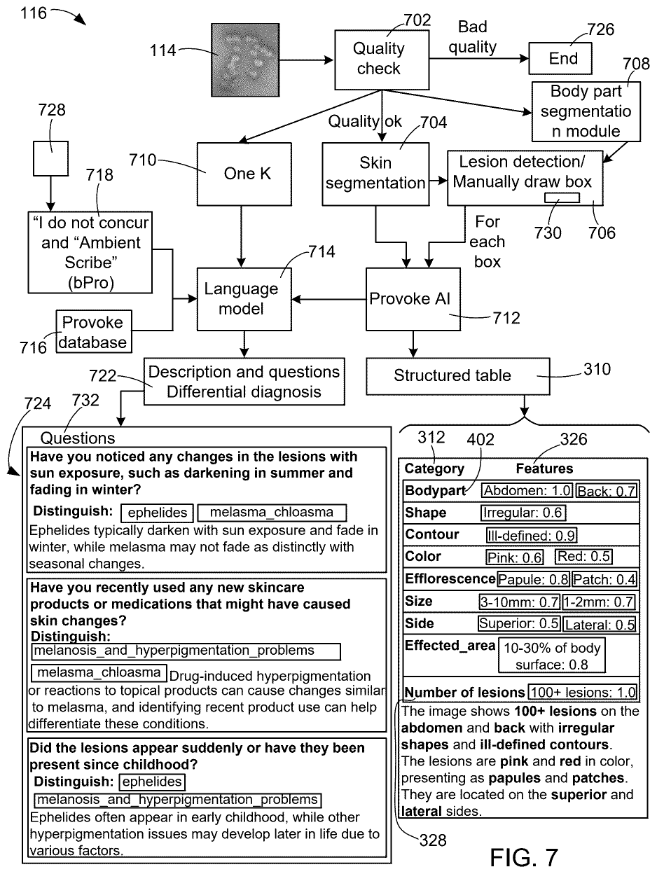

Referring now to FIG. 7, therein is shown a flow overview of the image diagnostic mechanism 116 of FIG. 1 along with an example output of a provoke AI module (table) and a description formed by a language model. For example, the flow includes an image quality check module 702, a skin segmentation module 704, a lesion detection module 706, a body part segmentation module 708, a differential diagnosis generation module 710, a provoke AI module 712, a language model module 714, a provoke database 716, a clinician input block 718, the structured table 310 with the category 312 and the feature 326, a description and questions output 722, a questions panel 724, and an end block 726.

By way of an example, the image quality check module 702 checks for image blur and lighting conditions. By way of an example, the skin segmentation module 704 explicitly targets affected skin regions 322 of FIG. 3. By way of an example, the lesion detection module 706 analyzes the acceptable patient image to detect and identify potential skin lesions 322 or ailments. By way of an example, the body part segmentation module 708 is a processing stage that labels anatomical zones within an image.

By way of an example, the differential diagnosis generation module 710 provides multiple potential conditions ranked by likelihood. By way of an example, the provoke AI module 712 is a post-processing stage that converts lesion and segmentation inputs into structured diagnostic tables and narrative descriptors. By way of an example, the language model module 714 is a text-generation engine that transforms structured data and clinician inputs into narrative descriptions and targeted questions. By way of an example, the provoke database 716 is a repository that stores disease templates, codes, and reference information for knowledge retrieval.

By way of an example, the clinician input block 718 is an interface element that captures user feedback and free-text comments. By way of an example, the description and questions output 722 is a composite message that presents narrative findings and follow-up queries generated by the compute system 100. By way of an example, the questions panel 724 is an on-screen region that displays system-generated queries for user response. By way of an example, the end block 726 is a terminal node that signifies completion of the processing sequence.

For example, this flow illustrates robust capabilities, supporting accurate, reliable, and clinically meaningful assessments through interactive and flexible workflows. The image quality check module 702 functions as a quality filter, classifying the patient images 114 of FIG. 2 as either acceptable or unacceptable based on various quality metrics, such as blurriness, bad luminosity (e.g., images too bright, too dark, or too noisy), and inappropriate image distance. As a specific example, the image quality check module 702 assesses metrics such as a blurry metric and a bad luminosity metric, comparing them against predefined thresholds to determine acceptability. The patient images 114 failing these criteria are flagged as unacceptable, prompting the user to retake and upload a higher-quality image.

For example, in the event that the image quality check module 702 classifies the patient image as unacceptable, the diagnostic mechanism 116 of FIG. 1 can terminate further analysis of the image. The compute system 100 of FIG. 1, the image diagnostic mechanism 116, or a combination thereof can then provide feedback or prompts to the user, indicating the specific quality issues identified, such as blurriness, inappropriate luminosity, or inadequate image distance. These informative prompts enable users to understand the deficiency in image quality clearly and guide them to retake and upload a new image that meets the necessary criteria for subsequent diagnostic processing. By proactively addressing image quality issues, the diagnostic mechanism helps ensure the reliability and clinical accuracy of the diagnostic outcomes.

For example, if the image quality check module 702 classifies the patient image 114 as acceptable, the patient image 114 proceeds along multiple paths. In one path, the patient image 114 is processed by the skin segmentation module 704, which identifies and isolates relevant skin regions 322 from non-skin regions. The image diagnostic mechanism 116 flows through the skin segmentation module 704 in an operation mode of real-time as well as non-real-time.

For example, the skin segmentation module 704 generates a skin region 322 based on a patient image 114. As an example, the image quality check module 702 accepts a patient image 114 captured in the user interface 202, passes the patient image 114 to the skin segmentation module 704. In this example, based on pixel-level classification, the skin segmentation module 704 produces a mask that contains only skin pixels, and this mask is the skin region 322.

For example, the image diagnostic mechanism 116 can include a separate path where the patient image 114 is processed by the lesion detection module 706 from the image quality check module 702. Additionally, a further path from the image quality check module 702 includes processing by the differential diagnosis generation module 710 (labeled One K), which generates a list of potential diseases identified by the disease 318. This list is then used to prompt the language model module 714 on how to describe detected lesions.

For example, the skin segmentation module 704 can be implemented using a neural-network architecture, such as a U-net model characterized by an encoder-decoder structure. As an example, the encoder portion decreases spatial dimensions at each layer while increasing channel depth, whereas the decoder portion inversely increases spatial dimensions while reducing channel depth. This architecture effectively captures fine details and spatial relationships within the skin images. As a specific example, an EfficientNet-based architecture, a convolutional neural network utilizing a compound scaling method, can also be employed. EfficientNet scales dimensions of depth, width, and resolution uniformly and can be pre-trained on large datasets such as ImageNet to enhance segmentation accuracy.

For example, the skin segmentation module 704 segments various skin regions including specific areas, such as eyes, nails, tattoos, and affected or suspected skin areas (e.g., psoriasis lesions, tumors). Optionally as an example, the segmentation includes scalp areas when clearly visible and unoccluded by hair. The skin segmentation module 704 effectively excludes irrelevant objects on the skin surface, such as clothing and jewelry, but continues to segment visible skin beneath transparent objects, like glasses. The resulting segmented images have non-skin regions removed or blacked out, thus minimizing background noise and ensuring diagnostic analysis focuses strictly on anatomical skin areas, enhancing the reliability and accuracy of subsequent dermatological assessments.

For example, the lesion detection module 706 (e.g. also lesion detection/manually-draw-box module) performs detailed object detection, identifying even small irritated skin regions, identifying skin disease or ailments, classifying the skin disease or ailments as detected lesions, or a combination thereof according to specific dermatological criteria, lesion types, or dermatological conditions. The image diagnostic mechanism 116 addresses common challenges such as poorly defined lesion borders and varying lesion-segmentation standards among clinicians by leveraging a carefully selected neural-network architecture and comprehensive training datasets. Additionally as an example, the lesion detection module 706 allows user-defined boxes, such as the bounding boxes 302 of FIG. 3, around skin regions of interest, enabling flexible and interactive selection of areas for further detailed assessment.

For example, the lesion detection module 706 identifies the region of interest 324 from the skin region 322. The masked output that includes the skin region 322 of the skin segmentation module 704 feeds the lesion detection module 706, which searches only the skin region 322, applies probability maps, and returns the bounding box 302 around each detected lesion, thereby establishing every region of interest 324.

For example, the lesion detection module 706 generates a lesion type 730 based on the region of interest 324. By way of an example, the lesion type 730 is a classification label that characterizes a detected dermatological finding. As an example, for every bounding box 302, the lesion detection module 706 classifies lesion morphology to generate a label and forwards that label as the lesion type 730 to both the differential diagnosis generation module 710 and the provoke AI module 712.

As an example, the lesion detection module 706 receives three context layers for every pixel patch the lesion detection module 706 analyses: (i) the skin-only mask from the skin segmentation module 704, (ii) the body-zone tag from the body part segmentation module 708, and (iii) the raw color-texture data from the acceptable patient image 114. As a specific example, inside the lesion detection module 706, a convolutional neural-network backbone-trained on thousands of dermatologist-labelled bounding boxes-extracts multi-scale attributes such as border sharpness, color variance, scaling patterns, and surface topology. In the example, a classification head then compares those attributes to prototype embeddings learned during training; the soft-max winner is emitted as the lesion type 730 (e.g., papule, plaque, macule). That label is attached to the bounding box 302, forwarded to the differential diagnosis generation module 710 to seed its disease ranking, and written into the lesion row of the structured table 310 generated by the provoke AI module 712.

For example, the lesion detection module 706 generates the bounding box 302 around the region of interest 324 in the skin region 322. In this example, the skin segmentation module 704 first masks non-skin pixels, thereby producing the skin region 322. The masked image is then sent to the lesion detection module 706, which scans only the skin region 322 and encloses each high-probability lesion cluster within the bounding box 302. Because every bounding box 302 lies substantially inside the skin region 322, the lesion detection module 706 identifies the region of interest 324 from the skin region 322 by generating the bounding box 302 around the region of interest 324 in the skin region 322.

For example, the body part segmentation module 708 operates after the image quality check module 702 has accepted an image and the skin segmentation module 704 has masked out non-skin pixels. Using that clean skin mask, the body part segmentation module 708 assigns every skin region to anatomical zones-arm, leg, trunk, face, scalp, and so on-producing a label map that accompanies the image downstream. Those body-zone tags flow directly into the lesion detection module 706, which now generates each bounding box 302 with a built-in attribute for the body part 402 and prunes proposals that cross zone boundaries.

For example, the differential diagnosis generation module 710 generates the possible disease classes 306 for the lesion type 730 for reviewing by a clinician 728. In this example, the lesion detection module 706 first classifies each bounding box 302 and produces the corresponding lesion type 730. The lesion type 730, together with auxiliary features such as the body part 402 from the body part segmentation module 708 and color-texture vectors from the patient images 114, is forwarded to the differential diagnosis generation module 710.

Continue with the example, the differential diagnosis generation module 710 consults an internal disease-feature matrix and ranks multiple candidate conditions, emitting the list as the possible disease classes 306, which constitutes a differential diagnosis output. This ranked list is appended to the structured table 310 and is simultaneously displayed in the description and questions output 722 on the user interface 202. The clinician 728 reviews the possible disease classes 306 in real time, optionally flags disagreements (e.g., “I do not concur”) via the clinician input block 718.

As an example, the possible disease classes 306 constitute the differential diagnosis output since the possible disease classes 306 contain several alternative conditions that can account for the observed lesion type 730, each paired with the likelihood score 320, to present multiple, ranked possibilities so that the clinician 728 can compare and rule out entries rather than rely on a single label for the diseases 318. The differential diagnosis generation module 710 generates this list specifically for the lesion in question, so the output is tailored to the presentation of the patient 112.

For example, the same tags feed the provoke AI module 712, which writes the same tags into a body part row of the body part 402 of the structured table 310. Because the language model module 714 reads that table, every narrative the language model module 714 emits in the description and questions output 722 starts with the correct anatomical site, and the questions panel 724 can pose zone-specific follow-up queries. In turn, the anatomically precise boxes and descriptors sharpen the ranked disease list produced by the differential diagnosis generation module 710, reducing clinician ambiguity and bolstering diagnostic accuracy across the entire flow.

For example, the provoke AI module 712 generates the structural language output 316 with the possible disease class 306 for the bounding box 302. For each bounding box 302, the lesion detection module 706 forwards the corresponding lesion type 730 to the differential diagnosis generation module 710, which selects and ranks candidate diseases 318. Each of the diseases 318 and the likelihood score 320 corresponding to each disease 318 are returned to the provoke AI module 712 and entered in the possible disease class column of the structured table 310. The language model module 714 then converts the structured table 310 into the structural language output 316 shown on the user interface 202, so that the narrative displayed for every bounding box 302 contains the possible disease classes 306.

For example, the provoke AI module 712 generates the structural language output 316 with the feature 326 corresponding to the category 312. In the example, the provoke AI module 712 fills every row of the structured table 310 with concrete values of the feature 326 that correspond one-to-one with the category 312. For example, because each recorded value of the feature 326 is a data point derived directly from the lesion pixels, every feature inherently corresponds to the respective category 312. When the language model module 714 converts the structured table 310 into the description and questions output 722, each sentence incorporates those same feature values of the feature 326 so the generated structural language output 316 explicitly contains the feature 326 corresponding to the category 312.

For example, the structural language output 316 can include the description and questions output 722, the structured table 310, or a combination thereof. Also for example, the description and questions output 722 is the composite payload produced by the language model module 714 after the language model module 714 ingests three key inputs: the PROVOKE-based table, such as the structured table 310, from the provoke AI module 712, the likelihood-ranked disease list from the differential diagnosis generation module 710, and any free-text or checkbox feedback captured in the clinician input block 718.

Continuing the example, within that payload, a narrative section explains every one of the bounding box 302 in clinically formatted prose-drawing on body-zone tags from the body part segmentation module 708—while an embedded section of a questionnaire 732 enumerates follow-up queries that help distinguish among the candidate diseases. As an example, the questionnaire 732 is routed directly to the questions panel 724 on the user interface 202, where the clinician 728 can respond in real time; those responses flow back into the clinician input block 718, enabling the language model module 714 to update or expand the description and questions output 722 in an iterative feedback loop before the workflow terminates at the end block 726. For example, the flow diagram concludes at the end block 726, representing logical termination of the diagnostic pipeline.

For example, the provoke AI module 712 generates the structural language output 316 with the category 312 including the number of lesions 328. In this example, the lesion detection module 706 counts how many discrete lesions fall within each bounding box 302 and forwards that count to the provoke AI module 712. The Provoke AI module 712 records the count for the number of lesions 328, as one of the categories 312 inside the structured table 310 to be displayed for the structural language output 316. Also for example, in addition to the number of lesions 328, the category 312 includes body part 402, arrangement, shape, contour, color, efflorescence, size, side, affected area, as examples.

By way of an example, the questionnaire 732 is a set of follow-up queries generated for clinician review. For example, the language model module 714 can generate the questionnaire 732 based on the lesion type 730 for a clinician 728 to distinguish between different diseases 318.

For example, both the possible disease classes 306 and the structured table 310 are input into the language model module 714. Using Retrieval-Augmented Generation (RAG), the language model module 714 queries the provoke database 716 for distinguishing clinical features-color changes, distribution patterns, symptom timelines-based on the lesion type 730 and the top entries in the possible disease classes 306. By way of an example, RAG refers to a method that combines large language models (LLMs) with specialized medical knowledge bases to improve diagnostic accuracy, treatment planning, clinical decision support, or a combination thereof. As an example, the language model module 714 then formulates a set of yes/no or short-answer prompts that separate the candidate diseases, such as the possible diseases 318, in practice. The generated prompt list forms the questionnaire 732, which the questions panel 724 displays on the user interface 202.

Continue with the example, the clinician 728 reads each prompt displayed for the questionnaire 732 in the questions panel 724 and records responses directly within the questions panel 724. As a specific example, the questionnaire 732 in the questions panel 724 is “Have you noticed any changes in the lesions with sun exposure, such as darkening in summer and fading in winter?”, “Have you recently used any new skincare products or medications that might have caused skin changes?”, or “Did the lesions appear suddenly or have they been present since childhood?”. In the example, the clinician input block 718 forwards the collected responses from the clinician 728 to the language model module 714. The language model module 714 removes unlikely diseases from the possible disease classes 306 and updates both the narrative portion of the description and questions output 722 and the structured table 310.

It has been unexpectedly discovered that the clinician 728 therefore leverages the questionnaire 732—generated from the lesion type 730 and the associated disease spectrum in the possible disease classes 306—to refine differential diagnoses and achieve a more accurate final assessment.

For example, identified skin regions from the skin segmentation module 704 and lesions detected by the lesion detection module 706 independently or collectively provide inputs to the provoke AI module 712. In this embodiment, the lesion detection module 706 provides “for each box” output, whereby each detected lesion's box, such as the bounding box 302, is individually processed by the provoke AI module 712. The process can iterate for each of the bounding boxes 302 if more than one exists or can be performed concurrently or some combination thereof.

For example, the provoke AI module 712 pinpoints specific areas for analysis, correlating the segmented skin regions with identified lesions to localize and characterize dermatological findings individually for each lesion. The provoke AI module 712 generates the structured table 310, such as those shown in FIGS. 4 and 5, and corresponding detailed clinical descriptions.

For example, the clinical descriptions include specific dermatological attributes such as lesion shape, color, arrangement, distribution, and type, allowing precise clinical understanding and evaluation. As a specific example, for each annotated lesion box, such as the bounding box 302, the provoke AI module 712 extracts structured information of the selected area: Body part, Arrangement, Shape, Contour, Efflorescence, Size, Number of lesions, and Affected area.

For example, outputs from the provoke AI module 712, along with the differential-diagnosis outputs from the differential diagnosis generation module 710, are provided as inputs to the language model module 714. The language model module 714 generates comprehensive lesion descriptions and clinical diagnostic outcomes, articulated in a clear and medically relevant narrative form. These outputs include structured summaries and detailed interpretations of dermatological findings, facilitating effective communication among healthcare providers. These outcomes, further enriched with additional data generated through analytical pathways, are combined with diagnostic information stored in a provoke database 716, resulting in detailed and clinically meaningful diagnostic outcomes. For example, these outcomes include differential diagnoses listing potential dermatological conditions linked to the analyzed regions or detected lesions, enhancing clinical decision-making and diagnostic transparency.

For example, the provoke AI module 712 generates the structural language output 316 based on the category 312. As an example, the provoke AI module 712 first combines three inputs associated with each bounding box 302: the lesion type 730 supplied by the lesion detection module 706, the body-zone tag from the body part segmentation module 708, and pixel-scale measurements taken directly from the skin region 322. In this example, using a ruleset that maps those inputs to the PROVOKE schema, the provoke AI module 712 fills one row of the structured table 310, populating every category 312 field—the body part 402, arrangement, shape, contour, efflorescence, size, number of lesions, and affected-area. In that row, the lesion type 730 determines multiple downstream categories: for example, a lesion type classified as papule sets “Efflorescence=papule,” constrains permissible Shapes to “round” or “oval,” and influences Arrangement defaults such as “discrete.”

Continuing the example, the fully populated table, such as the structured table 310, is then passed to the language model module 714, which reads each category 312 value and emits a grammatically coherent paragraph; that narrative, together with the structured table 310 itself, constitutes the structural language output 316. For example, because every sentence in the narrative is auto-generated based on the underlying category values, and those categories were chosen according to the lesion type 730, the structural language output 316 remains strictly grounded in, and traceable back to, the original lesion morphology identified by the lesion detection module 706.

For example, the provoke AI module 712 merges the lesion type 730, a body part tag, such as the body part 402, from the body part segmentation module 708, and the skin mask from module 704 to fill PROVOKE categories, such as the categories 312, in the structured table 310. In this example, the language model module 714 converts the structured table 310 into narrative prose and a follow-up questionnaire, which the description and questions output 722 presents on the first device 102 through the user interface 202 to assist the clinician 728 in diagnosis.

For example, the provoke AI module 712 generates the structural language output 316 based on the body part 402, the skin region 322, and the lesion type 730. The provoke AI module 712 assembles the structured table 310, and each row of the structured table 310 is indexed by a specific category 312, namely the fields including the body part 402, arrangement, shape, contour, efflorescence, size, number of lesions, and affected-area. To populate those fields, the provoke AI module 712 draws three inputs: the body part 402 from the body part segmentation module 708, pixel measurements derived from the skin region 322 created by the skin segmentation module 704, and the lesion type 730 supplied by the lesion detection module 706. Because the narrative that the language model module 714 produces in the description and questions output 722 is a direct transformation of the table row, every sentence in the structural language output 316 is therefore based on the trio of data elements including the body part 402, the skin region 322, and the lesion type 730 when filling the corresponding category 312.

For example, the inputs to the language model module 714 include a list of differential diagnoses from the differential diagnosis generation module 710, the structured table 310 from the provoke AI module 712, information from a diagnostic-application user interface (upper left quadrant in FIG. 7), such as the clinician input block 718, where a clinician can check an “I do not concur” box, and Ambient Scribe text data captured during consultation, along with entries from the provoke database 716. The provoke database 716 can include, for each disease, information following PROVOKE settings, International Statistical Classification (ICD-10) codes, disease frequencies, differential diagnoses, additional information, or a combination thereof. For example, the provoke AI module 712 is trained using data augmentation operations LongestMaxSize, PadIfNeeded, HorizontalFlip, VerticalFlip, RandomRotate90, RandomBrightnessContrast, and GaussianBlur, and employs an EfficientNet-V2-M backbone trained in two folds with cross-validation.

For example, an input to the provoke AI module 712 is an image accompanied by bounding box annotations, such as those of the bounding box 302, that indicate the locations of skin conditions. As an example, in the training dataset, data augmentation is performed on both the image and a corresponding binary mask, where pixels inside the bounding boxes 302 are set to 1 and those outside are set to 0. As a specific example, after augmentation, the binary mask is applied to the image using the following example rule: pixel values inside the bounding boxes are kept unchanged (multiplied by 1), while those outside are scaled by 0.3 to keep blurred context. The example rule encourages the provoke AI module 712 to focus on the regions inside the boxes, while still retaining contextual information from outside the boxes in a reduced, “blurred” form. For example, the provoke database 716 stores, for each dermatological disease, a row including a disease-name field, an ICD-10 code field, a frequency-value field, a canonical PROVOKE-template field encoded as eight integers, and an exemplar URI field linking to reference images.

For example, the flow proceeds through input-data collection, disease matching, knowledge retrieval, description generation via Retrieval-Augmented Generation (RAG), and differential-question generation, with clinician interaction occurring in the questions panel 724. For example, diseases are matched to the input table, such as the structured table 310, knowledge is retrieved, and questions are generated to help differentiate between similar diagnoses.

For example, the clinician input block 718 serves as the touchpoint where the user feeds real-time feedback back into the pipeline. As an example, the clinician input block 718 receives the follow-up questions displayed in the questions panel 724, captures checkbox actions such as “I do not concur”, and stores free-text notes transcribed by Ambient Scribe. Those inputs travel directly to the language model module 714, which incorporates the inputs-along with the structured table 310 from the provoke AI module 712 and the ranked disease list from the differential diagnosis generation module 710—to refine the narrative in the description and questions output 722.

For example, because the clinician input block 718 also writes each confirmation or override to the provoke database 716, subsequent cases benefit from accumulated expert feedback. As an example, this loop ensures that upstream elements, including the body part segmentation module 708 and the lesion detection module 706, receive updated priors whenever the pipeline in the image diagnostic mechanism 116 processes similar images in the future.

It has been unexpectedly discovered that combining quality-based image triage in the image quality check module 702 with concurrently the skin segmentation module 704 and the lesion detection module 706 paths feeds cleaner, context-rich data to the provoke AI module 712 and the language model module 714, thereby improving diagnostic accuracy and reducing overall inference time while maintaining flexible clinician feedback via the clinician input block 718 and the questions panel 724.

For example, as specific examples depicted in FIG. 7, the image quality check module 702 includes checks for image blur and lighting conditions, while the skin segmentation module 704 explicitly targets affected skin regions. For example, differential diagnosis generation provides multiple potential conditions ranked by likelihood, and diagnostic evaluation stages illustrate potential interaction points where clinicians may input observations or preferences. For example, this flow illustrates robust capabilities, supporting accurate, reliable, and clinically meaningful assessments through interactive and flexible workflows.

For example, the image quality check module 702 functions as a quality filter, classifying the patient images as either acceptable or unacceptable based on various quality metrics, such as blurriness, bad luminosity (e.g., images too bright, too dark, or too noisy), and inappropriate image distance. For example, as a specific example, the image quality check module 702 assesses metrics such as a blurry metric and a bad luminosity metric, comparing them against predefined thresholds to determine acceptability. For example, the patient images 114 failing these criteria are flagged as unacceptable, prompting the user to retake and upload a higher-quality image.

For example, in the event that the image quality check module 702 classifies the patient image 114 as unacceptable, the image diagnostic mechanism 116 can terminate further analysis of the patient image 114. For example, the compute system 100, the image diagnostic mechanism 116, or a combination thereof can then provide feedback or prompts to the user 112, indicating the specific quality issues identified, such as blurriness, inappropriate luminosity, or inadequate image distance. For example, these informative prompts enable users 112 to understand the deficiency in image quality clearly and guide them to retake and upload a new image that meets the necessary criteria for subsequent diagnostic processing. For example, by proactively addressing image quality issues, the image diagnostic mechanism 116 helps ensure the reliability and clinical accuracy of the diagnostic outcomes.

For example, if the image quality check module 702 classifies the patient image 114 as acceptable, the patient image 114 proceeds in this example embodiment along multiple paths. For example, in one path, the patient image 114 can be processed by a skin segmentation module 704, which identifies and isolates relevant skin regions from non-skin regions. For example, the image diagnostic mechanism 116 flows through the skin segmentation module 704 in an operation mode of real-time as well as non-real-time. For example, in this example embodiment, the image diagnostic mechanism 116 can include a separate path where the patient image 114 from the image quality check module 702 can be processed by the lesion detection module 706, the body part segmentation module 708, or a combination thereof. For example, additionally, a further path from the image quality check module 702 includes processing by a differential diagnosis generation module 710, labeled “One K,” which generates a list of potential diseases. For example, this list is then used to prompt a language model on how to describe detected lesions.

For example, the output of the provoke AI module 712 is a structured information table. For example, the provoke AI module 712 outputs the structured table 310 including the Body-part field, Arrangement field, Shape field, Contour field, Efflorescence field, Size field, Number-of-lesions field, and Affected-area field and further described below.

For example, as an example for the language model module 714, the inputs of the language model module 714 includes a list of differential diagnosis from the differential diagnosis generation module 710, the structured table 310 from provoke AI module 712, information from a diagnostic-application user interface (upper left quadrant in FIG. 7), such as the clinician input block 718, that can provide a user interface to the image diagnostic mechanism 116 or the compute system 100 for an input or entry for “I do not concur”-if doctor does not agree with result from the differential diagnosis generation module 710, they will check the “I do not concur” box- and “Ambient Scribe” which is the conversation between doctor and patient during the consultation, the provoke database 716, or a combination thereof.

For example, in this example, the provoke database 716 can include for each disease, information following PROVOKE settings, International Statistical Classification (ICD) codes, disease frequencies, differential diagnoses, additional information, or a combination thereof. For example, the output of the provoke AI module 712 includes clinical descriptions (lower right-hand side of FIG. 7), such as the structured table 310, diagnostic questions (lower left-hand side of FIG. 7), such as the description and questions output 722, a list of differential diagnoses such as from the questions panel 724, or a combination thereof. For example, as an example, the list of augmentation is the combination of the following: LongestMax-Size, PadIfNeeded, HorizontalFlip, VerticalFlip, RandomRotate90, Random-BrightnessContrast, RandomBrightnessContrast, GaussianBlur.

For example, as example outputs of the provoke AI module 712, the outputs can include four vectors of the body part 402, body part detail, range of number of lesions, and the rest, such as arrangement, shape, contour, efflorescence, size, or a combination thereof. For example, the output below the structured table 310 in FIG. 7 is an example of the output. For example, there are a number of ways to implement the provoke AI module 712. For example, an example implement is utilizing the EfficientNet V2 size M as backbone. For example, the provoke AI module 712 can be trained with 2 folds and then do cross validation.

For example, as an example of the language model module 714, the language model module 714 can process potential disease information and associated features to generate a clear, focused, and comprehensive clinical description. For example, the clinical description aids clinical experts by being easy to understand and supports explainable diagnosis. Additionally, the language model module 714 generates targeted questions to help differentiate between similar possible diagnoses, especially in cases where the structured table 310 cannot provide sufficient data.

In one embodiment, the structured table 310 stores, for each dermatological disease, a row including a disease-name field; an International Classification of Diseases, Tenth Revision (ICD-10) code field; a frequency-value field expressed, for example, as cases per 100,000 population; a canonical Provoke-template field encoded as eight integers ordered {body part field, arrangement field, shape field, contour field, efflorescence field, size field, the number of lesions 328 field, affected-area field}; and an exemplar Uniform Resource Identifier (URI) field containing one or more links to representative reference images. The eight element fields listed in the attribute-table portion of FIG. 7 correspond, respectively, to the body part field, arrangement field, shape field, contour field, efflorescence field, size field, the number of lesions 328 field, and affected-area field.

As an example, the flow works as follows input data, disease matching, knowledge retrieval, description generation, and differential question generation. The process of the flows can be delineated to different steps, functions from one to another can overlap or partitioned differently, some of the steps can be combined, or a combination thereof.

Regarding the input data of the flow as an example, the user display is shown in the lower left quadrant of FIG. 7 where a clinician 728 or a user 112 on the clinician 728 behalf can interact with the image diagnostic mechanism 116. Also for example the clinician 728 or a user 112 on the clinician 728 behalf can check the “I do not concur” box in user interface to an embodiment, indicating the clinician 728 does not agree with all the listed output of the differential diagnosis generation module 710, the image diagnostic mechanism 116 can procced with the structured table 310 and “Ambient scribe” text data as input. Otherwise, we take top 5 differential diagnosis of the differential diagnosis generation module 710 and the structured table 310 as well as “ambient scribe” text data as input.

Regarding the disease matching of the flow as an example, the image diagnostic mechanism 116 can identify the disease that is most similar to the input table, such as the structured table 310, based on the provoke database 716 and “Ambient scribe” text data, As an example for the disease matching, the image diagnostic mechanism 116 can identify potential disease by comparing the features from the structured table 310 with the entries in the provoke database 716. The process for disease matching can calculate the percentage of matching features and select diseases with a match score above a certain threshold.

Regarding the knowledge retrieval of the flow as an example, the image diagnostic mechanism 116 can utilize the diseases from the differential diagnosis generation module 710 and result from the process of disease matching and “ambient scribe” text data and query the provoke database 716 again to extract relevant medical knowledge.

Regarding the description generation of the flow as an example, the image diagnostic mechanism 116 can utilize the retrieved knowledge as context to generate a clinical description using Retrieval-Augmented Generation (RAG). As an example of RAG, the input to the language model module 714 is contextually enriched using disease-specific data, with additional text preprocessing applied to improve the relevance and accuracy of the generated responses.

Regarding the differential question generation of the flow as an example, the user display is shown in the upper left quadrant of FIG. 7 where a clinician 728 or a user 112 on the clinician 728 behalf can interact with the image diagnostic mechanism 116 with the questions. The image diagnostic mechanism 116 can utilize the diseases identified from the process of description generation to generate potential distinguishing questions using RAG and structured output, helping to differentiate between the possible diagnoses.

For example, FIG. 7 illustrates the sequential and concurrent integration of the image quality check module 702, the skin segmentation module 704, direct lesion or ailment detection by the lesion detection module 706, differential diagnosis generation by the differential diagnosis generation module 710, and diagnostic evaluation processes using the description and questions output 722. The workflow described underscores the diagnostic mechanism's robust capabilities, facilitating accurate, reliable, and clinically meaningful dermatological assessments while accommodating interactive user inputs and flexible diagnostic workflows.

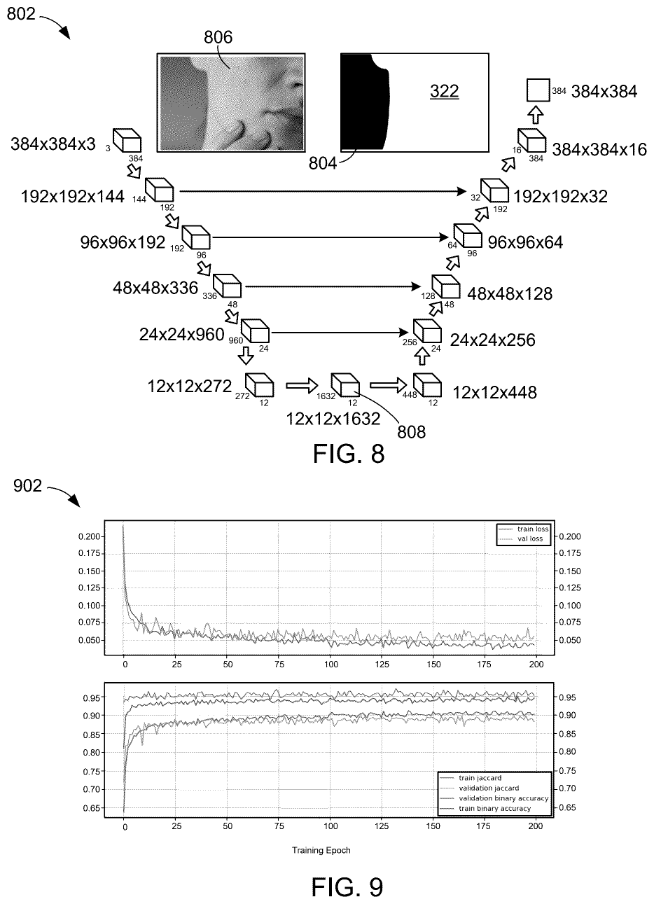

Referring now to FIG. 8, therein is shown an example of a processing flow 802 of the skin segmentation module 704 of FIG. 7 in an embodiment. As a specific example, FIG. 8 depicts an example of an implementation of the skin segmentation module 704 as a convoluted neural network with 508 layers with 25.7 million parameters.

Regarding the loss and metric to measure accuracy of the skin segmentation module 704, as an example, the metric to measure accuracy/performance of the model is Jaccard score. The Jaccard score measures the similarity between two sets of data to see which members are shared and distinct.

For example, regarding accuracy, the compute system 100 of FIG. 1, the image diagnostic mechanism 116 of FIG. 1, or a combination thereof can be trained for 200 epochs with 4516 images in each epoch, about 80% data set is reserved for training set and the rest for validation. To measure the accuracy of the model, the metric utilized is Jaccard score:

$$J ( A , B ) = \frac{\vert A ⋂ B \vert}{\vert A ⋃ B \vert} \qquad ( 5.1 )$$

The Jaccard score is a value between 0 and 1. The value of 0 indicates the data sets are completely different and a value of 1 indicates the data sets are identical.

As an example, the loss function of the skin segmentation module 704 can be defined as:

$$l ( y , \hat{y} ) = 1 - \frac{{\sum }_{i , j} {f}_{1}}{{\sum }_{i , j} {f}_{2}} where , \qquad ( 5.2 )$$

$${f}_{1} = 1 - {( 1 - {y}_{1} )}^{k} , {f}_{2} = {( 1 - {y}_{2} )}^{h} , {y}_{1} = y \hat{y} , {y}_{2} = ( 1 - y ) ( 1 - \hat{y} )$$

h=2.5, k=2.5, ŷ is the true label and y is the prediction. The sum in the formula of the loss function l is taken over two indices: index i stands for index of data sample {yᵢ}ᵢ, index j stands for the component of the vector label yᵢ=(yᵢⱼ)ⱼ. This loss considers not only the mask (y and ŷ) but also the inverse mask (1−y and 1−ŷ). By varying parameters h, k we could have many different versions of this loss function for different purposes. For example, if we reduce k and increase h, we will increase the priority area of pixels valued 1 (standing for the skin region 322) compared to area of pixels valued 0 (standing for a non-skin region 804). In this task, we take h=k=2.5 to somehow balance priority for the skin region 322 compared to the non-skin region 804. By way of an example, the non-skin region 804 is a background portion outside the region of interest 324 of FIG. 3 excluded from further analysis.

For example, the skin segmentation module 704 receives an input image 806 in a 384×384×3 format. The input image 806 is processed to reduce the input image 806 to a 192×192×144 format to divide the input image 806 into smaller segments in order to expose details of the input image 806. The differentiation of the skin region 322 and the non-skin region 804 are the result of the frame reduction process. The frame reduction process continues down to a target pixel array 808, such as the 12×12×1632 pixel array 411, then is processed through a convoluted neural network to increase the number of channels, before converging the data from 12×12×488 back to a single frame of 384×384 to differentiate the skin region 322 and the non-skin region 804.

Referring now to FIG. 9, therein is shown an example for performance graphs 902 of the skin segmentation module 704 of FIG. 7 in an embodiment. As described in FIG. 9, the metric used as an example is the Jaccard score. FIG. 9 shows the performance graphs 902, as an example, up to 200 epochs of training with 0.901 Jaccard score and binary accuracy is 0.962 for the skin segmentation module 704. FIG. 9 depicts the Jaccard score, binary accuracy and loss of the skin segmentation module 704.

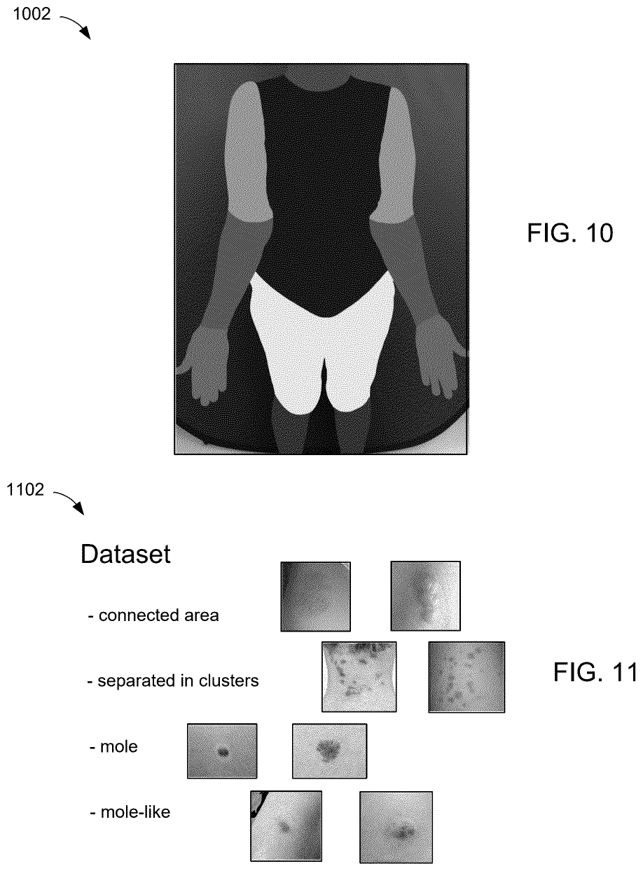

Referring now to FIG. 10, therein is shown an example of a task 1002 of the body part segmentation module 708 of FIG. 7 in an embodiment. For example, the task 1002 is body-part segmentation: starting with a full-body photograph, the body part segmentation module 708 isolates and labels anatomical body zones: head/neck, trunk, upper-arm, forearm, hand, thigh, shin, and foot, by assigning a distinct mask channel (e.g. rendered as a unique color shown in FIG. 10) to each zone. The task 1002 produces a pixel-accurate map that provides downstream components or modules which pixels belong to which body zone, so the lesion detection module 706 can crop, analyze, and describe lesions with anatomical context rather than processing the entirety of the patient image 114 of FIG. 1.

For example, the body part segmentation module 708 is designed to enhance the accuracy of the lesion detection module 706. As an example, the patient image 114 taken from a distance of 1 m-2 m can result in unsatisfactory prediction outcomes. To address this limitation, the body part segmentation module 708 is utilized to crop the body of the patient 112 of FIG. 1 into smaller pieces. By cropping the patient image 114 into specific body parts, the body part segmentation module 708 can focus on smaller areas, improving the precision of lesion detection.

For example, the body part segmentation module 708 can identify and localize specific regions of the human body within the patient image 114. The body part segmentation module 708 can enhance diagnostic precision by providing more accurate predictions, which is essential for applications in medical imaging and dermatological analysis.

For example, the body part segmentation module 708 segments the body parts even when the body parts are under other objects such as clothes, other body parts, hair, etc. so the segmented body parts retain anatomical fidelity despite occlusions.

For example, the data set contains about 5200 images equally distributed between men and women with diverse styles: entire body, with head, without the head, with clothes, and without clothes or underwear. This diversity underpins the robustness exhibited by the mask overlay.

For example, the body part segmentation module 708 includes data augmentation including HorizontalFlip, ShiftScaleRotate, GaussNoise, GaussianBlur, HueSaturation Value, CLAHE, and RandomBrightnessContrast. These augmentations improve generalization of the U-net whose encoder-decoder funnels are abstractly illustrated by the converging-diverging arrows surrounding the colored mask.

For example, the approach of the body part segmentation module 708 uses multi-channel segmentation. Each channel segmentation corresponds to a human part needed for segmentation. For illustrative purposes, the separate color layers in FIG. 10 depict those channels after soft-max fusion. For example, the body part segmentation module 708 includes a U-shaped architecture with an encoder-decoder scheme, an encoder that reduces the spatial dimensions, a decoder that increases the spatial dims while reducing the channels, and an EfficientNet-B4 encoder that substitutes for the left branch, as examples.

For example, loss that works the best for image segmentation of the body part segmentation module 708 is Lovasz loss, a differentiable surrogate of Intersection-Over-Union (IoU). However, we can smooth the hinge loss by replacing RELU by (ELU+1), where RELU (Rectified Linear Unit) serves as an activation function in neural-network layers architecture, which can improve both learning speed and model interpretability. Another trick is consideration of a symmetric Lovasz loss: consider not only a predicted segmentation and a provided mask but also the inverse prediction and the inverse mask (predict mask for negative case).

For example, the Intersection-Over-Union (IoU) score is used as the metric for the task 1002. As an example, the IoU score, also known as the Jaccard similarity coefficient, is a statistic used in understanding the similarities between sample sets. For the segmentation problem, the IoU score measures the number of common pixels between the target and prediction masks divided by the total number of pixels present across both masks.

For example, the loss calculation of the body part segmentation module 708 relies on the formula: IoU=target n prediction/target U prediction, where target n prediction is an area of overlap, and target U prediction is an area of union. As a specific example, the model attains an average IoU score=0.85 after five-fold cross-validation over 300 epochs, quantitatively validating the crisp region boundaries.

For example, to measure the performance of our model, we use the k-fold Cross-Validation technique to split our data. As a specific example, the data is randomly partitioned into 5 equal-sized subsamples. Of the five subsamples, a single subsample is retained as the validation data for testing the model, and the remaining 4 subsample is used as training data. For each fold, the model is trained in 300 epochs. The average IoU score is 0.85.

Referring now to FIG. 11, therein are shown examples of lesion classes 1102 detected by the lesion detection module 706 of FIG. 7 in an embodiment. For example, the lesion detection module 706 plays a pivotal role in dermatological practices and healthcare systems by offering precise identification and characterization of various skin abnormalities, including moles, melanomas, and other potential indicators of skin diseases. With the aid of advanced machine learning techniques, the lesion detection module 706 provides dermatologists and healthcare professionals with invaluable support with detecting lesions and classifying of skin conditions. By swiftly identifying suspicious lesions, the lesion detection module 706 enables timely intervention, potentially saving lives by facilitating early detection of skin diseases and other dermatological conditions.

For example, the lesion detection module 706 detects and classifies lesions with high precision, improving lesion image capture and diagnostic prediction accuracy. The lesion detection module 706 can utilize a nano model version that allows the system 100 of FIG. 1, the image diagnostic mechanism 116 of FIG. 1, or a combination thereof to run directly on the first device 102 of FIG. 1, the second device 106 of FIG. 1, or a combination thereof, ensuring real-time fast predictions. As an example, the lesion detection module 706 can utilize a nano model version that runs entirely on the first device 102 for real-time triage. As another example, a heavier server version couples skin segmentation and body part detection that run on the first device 102 and the second device 106 for higher accuracy. This approach minimizes latency and enhances the responsiveness of applications requiring immediate data processing. The on-device execution of the nano model not only optimizes performance but also enables efficient operation in dynamic environments. Consequently, this integration provides a significant improvement in the system's overall effectiveness.

For example, the training dataset includes 58904 images, consisting of skin lesions, normal skin samples, and no lesion data. The lesions fall into four categories: connected area, separated in clusters, mole, and mole-like. The lesion detection module 706 can accurately identify the location and type of various potential lesions within the patient image 114. The algorithm provides bounding box coordinates for each mole detected in every image, facilitating precise identification and analysis.

For illustrative purposes, examples of the four lesion classes 1102 that the lesion detection module 706 learns to detect are connected area, mole, mole-like, and separated in clusters. The classification is based on distinct morphological characteristics, enhancing the accuracy of lesion categorization.

For example, to improve the dataset's quality, lesion instances are annotated and re-annotated by expert annotators using our proprietary annotation system-TAS (Torus Annotation System). Simultaneously, an extensive deduplication process was undertaken to eliminate redundant entries. These measures are critical for enhancing data integrity and ensuring the reliability of future analyses. The refined dataset now provides a more accurate and robust basis for research applications.

For example, to address the issue of misclassifying non-skin areas (e.g., the non-skin region 804 of FIG. 8) as skin (e.g., the skin region 322 of FIG. 3), an additional type of bounding box specifically for skin regions is introduced. This enhancement aims to improve the model's ability to differentiate between skin and non-skin areas accurately. Furthermore, the model tends to ignore larger moles, likely due to their underrepresentation in the training dataset. To rectify this, macro images of skin cancer are added to the dataset, resized to smaller dimensions, and overlaid onto existing skin regions in the dataset. A skin segmentation module is employed to accurately identify and segment skin areas within images, ensuring precise placement of synthetic moles, tattoo augmentation, skin tone matching, darker skin augmentation, or a combination thereof. In cases where multiple skin regions are detected within a single patient image 114 of FIG. 1, the skin segmentation module 704 of FIG. 7 separates the skin regions into distinct bounding boxes to avoid overlapping and enhance segmentation clarity. The process of creating synthetic moles involves two key steps: 1) the skin areas are segmented, and their coordinates are saved; and 2) images of moles are superimposed onto these segmented skin areas in real-time.

Continue with the example, the box and segmentation labels for model training are formatted as following coordinates: x1, y1, x2, y2, . . . , xn, yn. This format facilitates the provision of precise skin segmentation points, which are utilized for augmentations involving moles and tattoos. This method increases the diversity of mole appearances in the dataset, thereby improving the model's ability to detect larger moles accurately. By implementing these strategies, the robustness and accuracy of the skin segmentation model are expected to improve significantly, addressing current limitations in non-skin area detection and mole recognition.

For example, to address the situation where the model detects as skin when there is no skin, we add padding of the skin color (before black padding was used). The Fitzpatrick skin type classification is a utilized system in dermatology for categorizing human skin color. This classification aids in predicting the skin's response to ultraviolet (UV) radiation exposure, which encompasses six distinct skin types, each characterized by specific phenotypic and phototypic attributes.

Continue with the example, Type I: Very fair skin, often with red or blond hair and blue or green eyes. Individuals with this skin type always burn and never tan when exposed to the sun. Type II: Fair skin, usually with blond or light brown hair and blue, green, or hazel eyes. These individuals typically burn easily and tan minimally. Type III: Medium skin tone, common among people with darker hair and brown eyes. These people may burn moderately and gradually achieve a light tan. Type IV: Olive or light brown skin, often found in individuals with dark hair and brown eyes. These individuals tend to burn minimally and tan to a moderate brown shade. Type V: Brown skin, frequently seen in individuals with dark hair and brown eyes. This skin type rarely burns and tans well to a dark brown color. Type VI: Dark brown or black skin, with individuals who almost never burn and tan very easily and deeply.

Continue with the example, the Fitzpatrick scale is instrumental in various dermatological practices, including skin cancer risk assessment, laser treatment planning, and cosmetic procedures. The Fitzpatrick scale also provides a foundational framework for research involving skin-related conditions and treatments, enabling more precise and personalized medical care.

For example, CutMix augmentation mixes two images by cutting a patch from one image and pasting it onto another. The labels are also mixed proportionally to the area of the patch. CutMix encourages the model to focus on less discriminative parts of objects, enhancing its localization abilities and reducing overfitting. CutMix has been shown to improve performance on various tasks, including image classification and object detection. As a specific example, Cutmix augmentation checks every time that the boxes' overlapping area is not bigger than 30% of the boxes, choose randomly image parts to crop and paste to another image.

For example, mosaic augmentation involves combining four different images into a single image by splitting them into quadrants and merging them. Each quadrant of the resulting image contains a portion of one of the original images. This technique significantly increases the diversity of the training data by exposing the model to varied contexts and object compositions. Mosaic augmentation helps in improving the model's robustness and ability to generalize by providing more diverse training samples. The mosaic technique includes: 1) stack 4 images with np.stack 2) change coordinates of all boxes 3) random resize with the random scale=(0.9, 1.1):−resize image−multiply boxes' coordinates with the scale 4) random crop to model size-update boxes' coordinates.

For example, the model that was trained using Weighted Sampler: precision, recall, mAP50 metrics are higher for this version and the training curves are better. The model detects lesions more precisely and can detect bigger mole-like lesions. However, sometimes the model detects nails, eyes, mouths as lesions. It can be solved if we add more normal skin images. As a specific example, for the moment, normal skin dataset includes only 300 images. Also, the model does not perform well on darker skin. For this we can use dark skin augmentation. To do: count skin colors ratio for the dataset dark skin augmentation if needed normal skin augmentation tattoo augmentation-darker skin-tattoo augmentation-remove overlapping boxes NMS agnostic True-add body part detection.

For example, we introduce mole augmentation to have more mole examples in the dataset. For this, we use skin segmentation put mole on skin only. Before that, we check the skin tone in order to have mole augmentation on the skin color. We use this augmentation for 20% of images in the dataset. We increase the representation of mole instances within our dataset through a technique “mole augmentation”. This method involves the application of skin segmentation to ensure that the artificial moles are placed exclusively on skin regions. Prior to the augmentation process, we perform an analysis of skin tone to ensure that the augmented moles blend seamlessly with the natural skin color. This augmentation process was applied to 20% of the images in our dataset, amounting to approximately 40,000 images.−mole augmentation−add skin tone to mole augmentation update labels for mole augmentation remove overlapping boxes of different classes (correct NMS algorithm function). The model works good but still needs to be improved. For example, it would be better to add moles only to the images with the same skin tone. For this we can use the new multi skin model.

For example, in order to improve training process, we implement different augmentation techniques: stack images, crop, resize, blackout. The augmentation techniques are done online during the training routine, and we also control the percentage of different types of images in every batch. To enhance the learning process, images of lesions, moles, normal skin, and oops images are stacked, with four images combined at a time. The lesion detection module 706 controls the percentage of lesions, moles, normal skin, and “oops” images in each training batch to ensure balanced representation.

For example, a dedicated folder or metadata is maintained to categorize each image as normal skin, oops, lesion, or mole, facilitating efficient data management and retrieval. During training, the patient images 114 of FIG. 1 are first resized to a random dimension, followed by cropping to ensure variability and augmentation. Subsequently, the patient images 114 are stacked to create composite images that enrich the training dataset. This methodology ensures a diverse and representative dataset, improving the model's ability to generalize and accurately detect lesions. Such an approach aims to mitigate overfitting and enhance the robustness of the detection model.

For example, the connected-area, cluster, mole, and mole-like thumbnails in FIG. 11 correspond respectively to four lesion types 730 of FIG. 7 produced by the lesion detection module 706. Each thumbnail originated as a bounding box 302 of FIG. 3 extracted from the dataset after the skin segmentation module 704 isolates the skin region 322. The resulting lesion type 730 is forwarded to the differential diagnosis generation module 710 of FIG. 7, producing the possible disease classes 306 of FIG. 3 that populate the structured table 310 of FIG. 3 and appear in the description and questions output 722 of FIG. 7 for review by the clinician 728 of FIG. 7.

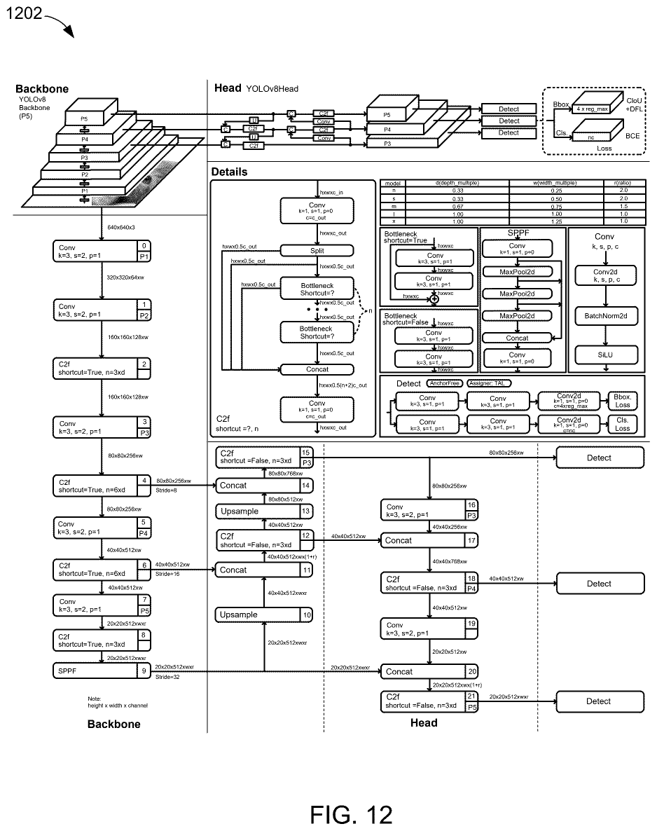

Referring now to FIG. 12, therein is shown an example of an architecture 1202 of the lesion detection module 706 of FIG. 7 in an embodiment. For example, the architecture 1202 includes the backbone-neck-head workflow that underpins real-time object detection for dermatological images.

For example, the architecture 1202 includes the YOLO (You Only Look Once) architecture, which is an object detection framework known for high speed and accuracy. Without employing a region proposal stage, YOLO frames detection as a single regression problem, directly predicting bounding boxes, such as the bounding box 302, the supplementary boxes 404, or a combination thereof, and class probabilities from full images in one evaluation run.

As an example, the architecture 1202 utilizes a convolutional neural network (CNN) to process images in a single pass, dividing the image into a grid and predicting bounding boxes and class probabilities for each grid cell simultaneously. This end-to-end approach significantly reduces computational complexity and enhances real-time performance. The improvements include the integration of anchor boxes, multi-scale predictions, and feature pyramid networks, which enhance the model's capability to detect objects at different scales and aspect ratios. YOLO's ability to balance speed and accuracy makes it particularly suitable for applications requiring real-time object detection, such as autonomous driving and video surveillance.

For example, the yolo-like model, such as the architecture 1202, was selected for the skin lesion detection task of the lesion detection module 706 due to real-time object detection capabilities and unified detection approach. This type of the architecture 1202 includes speed and accuracy. The model of the architecture 1202 includes several layers as followed.

For example, the model of the architecture 1202 includes the backbone, which is designed to extract features from the input image 114 of FIG. 1. The backbone includes multiple convolutional layers with various kernel sizes, strides, and activation functions to capture hierarchical feature representations. These layers form a deep convolutional neural network that processes the input image 114 at different scales, enabling the detection of objects of various sizes.

Also for example, the model of the architecture 1202 includes the neck, which includes a neck structure responsible for feature fusion. This is crucial for combining multi-scale information and improving the model's ability to detect objects of varying sizes. As an example, YOLOv8 uses a new architecture known as SPP-Net (Spatial Pyramid Pooling Network) and PANet (Path Aggregation Network) that enhances its ability to detect objects of different sizes and shapes.

Also for example, the model of the architecture 1202 includes the detection head, which is where the final object detection predictions are made. As an example, YOLOv8 retains the characteristic feature of the YOLO series, including the head. The head component generates predictions based on the features extracted by the backbone network and the neck architecture. The head includes layers that predict bounding boxes, such as the bounding box 302, the supplementary boxes 404, or a combination thereof, objectness scores, and class probabilities. The neck architecture uses anchor boxes to efficiently predict objects of different shapes and sizes.

For example, YOLO's architecture combines efficient feature extraction with precise object detection, enabling real-time inference on images and videos. The single-stage design and unified approach to object detection of the architecture 1202 is for various applications. As an example, the model structure of the architecture 1202 is built around a single convolutional neural network (CNN) that processes the entire image at once, enabling end-to-end object detection. The network divides the input image into an S×S grid, with each grid cell responsible for predicting a fixed number of bounding boxes and the associated confidence scores. The confidence score reflects the likelihood that the box contains an object and the accuracy of the predicted bounding box. Each bounding box prediction includes five components: the coordinates of the box center (x, y), width and height, and the confidence score.

For example, in operational context, the lesion detection module 706 of FIG. 7 receives skin-only imagery from the skin segmentation module 704, applies the YOLOv8 backbone-neck-head stack shown in FIG. 12, and outputs the bounding boxes 302, the supplementary boxes 404, or a combination thereof, together with the lesion type 730 scores. In the example, these predictions feed the possible disease classes 306 in the structured table 310, which are then included in the description and questions output 722 for the clinician 728 to review, achieving the latency and accuracy targets quantified when the skin segmentation module 704 is enabled.

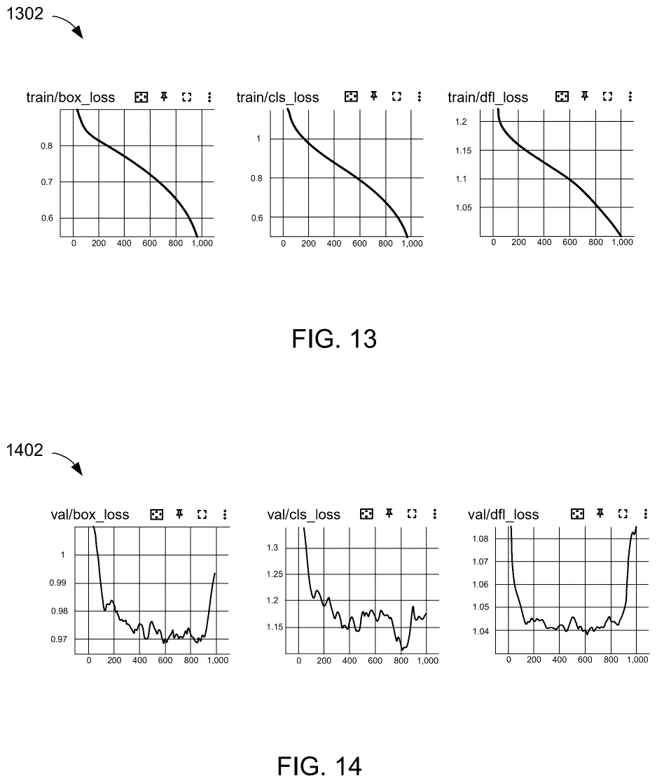

Referring now to FIG. 13, therein are shown examples of training performance graphs 1302 of the lesion detection module 706 of FIG. 7 in an embodiment. Each example graph tracks the behavior of a distinct loss component during the 1,000-epoch.

For example, in the left panel (train/box_loss), the x-axis represents consecutive training epochs (0→1,000) and the y-axis represents the bounding-box regression loss. The steadily falling trace illustrates the network's improving ability to localize lesion boxes precisely.

For example, in the middle panel (train/cls_loss), the x-axis represents consecutive training epochs (0→1,000), while the y-axis measures the classification error for lesion classes. The monotonic decline confirms that the backbone-neck-head stack shown in FIG. 12 is learning to assign the correct lesion type 730 labels.

For example, in the right panel (train/dfl_loss), the x-axis represents consecutive training epochs (0→1,000), and the distribution-focal loss on the y-axis. The curve's downward trend indicates finer calibration of bounding-box probability distributions, which in turn boosts the quality of the possible disease classes 306 that populate the structured table 310.

Referring now to FIG. 14, therein are shown examples of validation performance graphs 1402 of the lesion detection module 706 of FIG. 7 in an embodiment. The examples correspond to validation losses.

For example, each panel plots validation loss versus training epoch (0→1,000) for the YOLO-v8 nano network that underlies the lesion detection module 706. All validation curves are computed on validation: instances 12345 and instances 16617 of the skin-segmented dataset.

For example, in the left panel (val/box_loss), the x-axis shows epoch index and the y-axis shows the bounding-box regression loss. The curve initially peaks, falls toward a minimum near epoch 500, and rises again, indicating the point at which over-fitting begins if the skin segmentation module 704 is not supplied.

For example, in the middle panel (val/cls_loss), the x-axis shows epoch index and the y-axis shows lesion-class cross-entropy. The undulating trajectory illustrates how class-prediction accuracy improves, stabilizes, and then degrades slightly as the learning rate decays, mirroring the behavior of the lesion type 730 assignments.

For example, in the right panel (val/dfl_loss), the x-axis shows epoch index and the y-axis shows distribution-focal loss. The U-shaped trend tracks calibration quality of the bounding-box distributions that provide the possible disease classes 306 in the structured table 310.

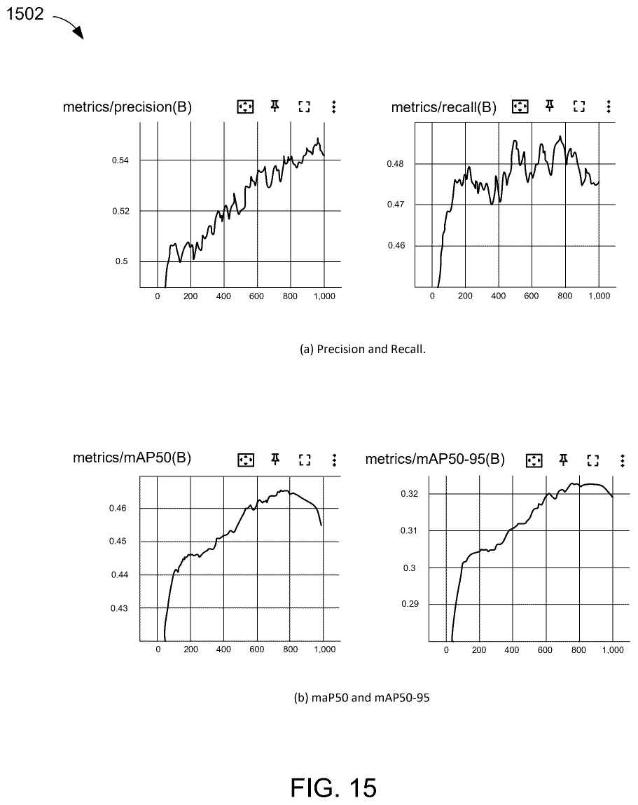

Referring now to FIG. 15, therein are shown examples of performance graphs 1502 of metrics during training of the lesion detection module 706 of FIG. 7 in an embodiment. The examples correspond to metrics during training.

For example, the upper-left panel (metrics/precision(B)) plots epoch index (0→1,000) on the x-axis against precision on the y-axis, where precision measures the proportion of predicted bounding boxes 302 that correctly match lesions detected in the validation set. For example, precision rises steadily from approximately 0.50 to approximately 0.55 as the nano YOLO-v8 model trains on 46,542 skin-segmented images, indicating improved positive-prediction accuracy.

For example, the upper-right panel (metrics/recall(B)) plots the same epoch range on the x-axis and recall on the y-axis, where recall quantifies the fraction of ground-truth lesions that the detector successfully localizes. For example, recall climbs from approximately 0.45 to approximately 0.48 before oscillating, showing that the skin segmentation module 704 of FIG. 7 and the lesion detection module 706 of FIG. 7 increasingly capture true lesions while balancing false negatives.

For example, the lower-left panel (metrics/mAP50(B)) presents mean Average Precision at 0.50 IoU versus epochs. The curve rises rapidly before plateauing near approximately 0.465, confirming that bounding-box localization accuracy at the 50% IoU threshold improves as weight updates propagate through the YOLO backbone-neck-head architecture 1202 of FIG. 12.

For example, the lower-right panel (metrics/mAP50-95 (B)) shows mean Average Precision averaged over IoU thresholds from 0.50 to 0.95. The curve ascends to approximately at or higher than 0.32, demonstrating robust multi-threshold performance that underpins reliable generation of the lesion types 730 of FIG. 7, the possible disease classes 306 of FIG. 3, and the structured table 310 of FIG. 3 presented to the clinician 728 of FIG. 7.

It has been unexpectedly discovered that monitoring precision, recall, mAP50, and mAP50-95 concurrently enables early-stopping at the epoch range where all four metrics plateau, thereby freezing weights that maximize both localization and classification quality while minimizing over-fitting, resulting in an outcome that directly enhances the accuracy of the description and questions output 722 of FIG. 7 delivered through the compute system 100 of FIG. 1.

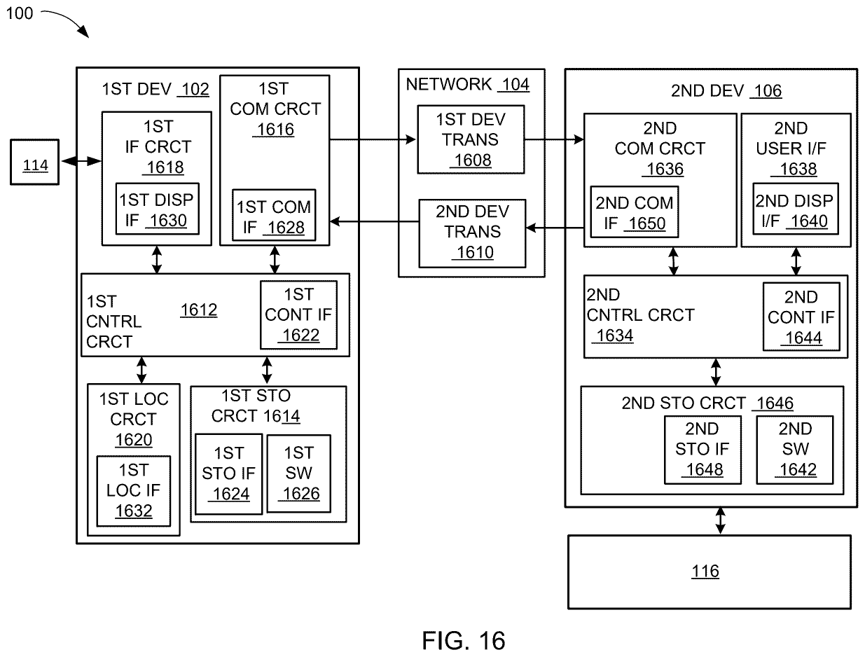

Referring now to FIG. 16, therein is shown an exemplary block diagram of the compute system 100 in an embodiment. The compute system 100 shown in this figure can be utilized to implement the embodiment in one of the devices or distributed or spread across multiple of the devices. The compute system 100, a portion of the compute system 100, or a combination thereof can execute the detection mechanism 116.

The compute system 100 can include the first device 102, the network 104, and the second device 106. The first device 102 can send information in a first device transmission 1608 over the network 104 to the second device 106. The second device 106 can send information in a second device transmission 1610 over the network 104 to the first device 102.

For illustrative purposes, the compute system 100 is shown with the first device 102 as a client device, although it is understood that the compute system 100 can include the first device 102 as a different type of device.

Also, for illustrative purposes, the compute system 100 is shown with the second device 106 as a server, although it is understood that the compute system 100 can include the second device 106 as a different type of device. For example, the second device 106 can be a client device. By way of an example, the compute system 100 can be implemented entirely on the first device 102 with some functions of the detection mechanism 116 executed by a first control circuit 1612.

Also, for illustrative purposes, the compute system 100 is shown with interaction between the first device 102 and the second device 106. However, it is understood that the first device 102 can be a part of or the entirety of an autonomous vehicle, a smart vehicle, or a combination thereof. Similarly, the second device 106 can similarly interact with the first device 102 representing the autonomous vehicle, the intelligent vehicle, or a combination thereof.

For brevity of description in this embodiment of the present invention, the first device 102 will be described as a client device, and the second device 106 will be described as a server device. The embodiment of the present invention is not limited to this selection for the type of devices. The selection is an example of an embodiment of the present invention.

The first device 102 can include a first control circuit 1612, a first storage circuit 1614, a first communication circuit 1616, a first interface circuit 1618, and a first location circuit 1620. The first control circuit 1612 can include a first control interface 1622. The first control circuit 1612 can execute a first software 1626 to provide the intelligence of the compute system 100.

The first control circuit 1612 can be implemented in a number of different manners. For example, the first control circuit 1612 can be a processor, an application specific integrated circuit (ASIC) an embedded processor, a microprocessor, a hardware control logic, a hardware finite state machine (FSM), a digital signal processor (DSP), or a combination thereof. The first control interface 1622 can be used for communication between the first control circuit 1612 and other functional units or circuits in the first device 102. The first control interface 1622 can also be used for communication that is external to the first device 102. The first control circuit 1612 can process information associated with the patient image 114 and execute portions of the detection mechanism 116.

The first control interface 1622 can receive information from the other functional units/circuits or from external sources, or can transmit information to the other functional units/circuits or to external destinations. The external sources and the external destinations refer to sources and destinations external to the first device 102.

The first control interface 1622 can be implemented in different ways and can include different implementations depending on which functional units/circuits or external units/circuits are being interfaced with the first control interface 1622. For example, the first control interface 1622 can be implemented with a pressure sensor, an inertial sensor, a microelectromechanical system (MEMS), optical circuitry, waveguides, wireless circuitry, wireline circuitry, or a combination thereof.

The first storage circuit 1614 can store the first software 1626. The first storage circuit 1614 can also store the relevant information, such as data representing incoming patient images 114, the detection mechanism 116 and the other embodiments, or a combination thereof.

The first storage circuit 1614 can be a volatile memory, a nonvolatile memory, an internal memory, an external memory, or a combination thereof. For example, the first storage circuit 1614 can be a nonvolatile storage such as non-volatile random-access memory (NVRAM), Flash memory, disk storage, or a volatile storage such as static random-access memory (SRAM).

The first storage circuit 1614 can include a first storage interface 1624. The first storage interface 1624 can be used for communication between the first storage circuit 1614 and other functional units or circuits in the first device 102. The first storage interface 1624 can also be used for communication that is external to the first device 102.

The first storage interface 1624 can receive information from the other functional units/circuits or from external sources or can transmit information to the other functional units/circuits or to external destinations. The external sources and the external destinations refer to sources and destinations external to the first device 102. The first storage interface 1624 can receive input from and source data to the detection mechanism 116.

The first storage interface 1624 can include different implementations depending on which functional units/circuits or external units/circuits are being interfaced with the first storage circuit 1614. The first storage interface 1624 can be implemented with technologies and techniques similar to the implementation of the first control interface 1622.

The first communication circuit 1616 can enable external communication to and from the first device 102. For example, the first communication circuit 1616 can permit the first device 102 to communicate with the second device 106 and the network 104. The first communication circuit 1616 can interact with the second device 106 for implementing the detection mechanism 116.

The first communication circuit 1616 can also function as a communication hub allowing the first device 102 to function as part of the network 104 and not limited to be an endpoint or terminal circuit to the network 104. The first communication circuit 1616 can include active and passive components, such as microelectronics or an antenna, for interaction with the network 104.

The first communication circuit 1616 can include a first communication interface 1628. The first communication interface 1628 can be used for communication between the first communication circuit 1616 and other functional units or circuits in the first device 102. The first communication interface 1628 can receive information from the second device 106 for distribution to the other functional units/circuits or can transmit information to the other functional units or circuits.

The first communication interface 1628 can include different implementations depending on which functional units or circuits are being interfaced with the first communication circuit 1616. The first communication interface 1628 can be implemented with technologies and techniques similar to the implementation of the first control interface 1622.

The first interface circuit 1618 allows the user 112 of FIG. 1 to interface and interact with the first device 102. The first interface circuit 1618 can include an input device and an output device. Examples of the input device of the first interface circuit 1618 can include a keypad, a touchpad, soft-keys, a keyboard, a microphone, a camera, an infrared sensor for receiving remote signals, or any combination thereof to provide data and communication inputs, such as the patient image 114. The first interface circuit 1618 can receive the patient image 114 provided by the patient 112 that can be manipulated by the first control circuit 1612.

The first interface circuit 1618 can include a first display interface 1630. The first display interface 1630 can include an output device. The first display interface 1630 can include a projector, a video screen, a touch screen, a speaker, a microphone, a keyboard, and combinations thereof. The first display interface 1630 can allow the patient 112 to view the results of the detection mechanism 116 and the other embodiments on the output device.

The first control circuit 1612 can operate the first interface circuit 1618 to display information generated by the compute system 100 and receive input from the user 112. The first control circuit 1612 can also execute the first software 1626 for the other functions of the compute system 100, including receiving location information from the first location circuit 1620. The first control circuit 1612 can further execute the first software 1626 for interaction with the network 104 via the first communication circuit 1616. The first control circuit 1612 can operate portions or all of the detection mechanism 116.

The first control circuit 1612 can also receive location information from the first location circuit 1620. The first control circuit 1612 can operate the detection mechanism 116 or portions thereof. The first control circuit 1612 can operate on information associated with the patient image 114, as well as any of the output for the detection mechanism 116 and any of the embodiments for display to the patient 112.

The first location circuit 1620 can be implemented in many ways. For example, the first location circuit 1620 can function as at least a part of the global positioning system, an inertial compute system, a cellular-tower location system, a gyroscope, or any combination thereof. Also, for example, the first location circuit 1620 can utilize components such as an accelerometer, gyroscope, or global positioning system (GPS) receiver.

The first location circuit 1620 can include a first location interface 1632. The first location interface 1632 can be used for communication between the first location circuit 1620 and other functional units or circuits in the first device 102.

The first location interface 1632 can receive information from the other functional units/circuits or from external sources or can transmit information to the other functional units/circuits or to external destinations. The external sources and the external destinations refer to sources and destinations external to the first device 102. The first location interface 1632 can receive the global positioning location from the global positioning system (not shown).

The first location interface 1632 can include different implementations depending on which functional units/circuits or external units/circuits are being interfaced with the first location circuit 1620. The first location interface 1632 can be implemented with technologies and techniques similar to the implementation of the first control circuit 1612.

The second device 106 can be optimized for implementing an embodiment of the present invention in a multiple device embodiment with the first device 102. The second device 106 can provide the additional or higher performance processing power compared to the first device 102. The second device 106 can include a second control circuit 1634, a second communication circuit 1636, a second user interface 1638, and a second storage circuit 1646.

The second user interface 1638 allows an operator (not shown) to interface and interact with the second device 106. The second user interface 1638 can include an input device and an output device. Examples of the input device of the second user interface 1638 can include a keypad, a touchpad, soft-keys, a keyboard, a microphone, or any combination thereof to provide data and communication inputs. Examples of the output device of the second user interface 1638 can include a second display interface 1640. The second display interface 1640 can include a display, a projector, a video screen, a speaker, or any combination thereof.

For example, the first display interface 1630, the second display interface 1640, or a combination thereof can be used to implement the user interface of FIGS. 2-6. As an example, the first display interface 1630, the second display interface 1640, or a combination thereof can be used to display the examples of the user interface 202 described in any of FIG. 2-6 or a combination thereof.

The second control circuit 1634 can execute a second software 1642 to provide the intelligence of the second device 106 of the compute system 100. The second software 1642 can operate in conjunction with the first software 1626. The second control circuit 1634 can provide additional performance compared to the first control circuit 1612. The second control circuit 1634 can execute instructions to implement all or some of the functions of the detection mechanism 116.

The second control circuit 1634 can operate the second user interface 1638 to display information. The second control circuit 1634 can also execute the second software 1642 for the other functions of the compute system 100, including operating the second communication circuit 1636 to communicate with the first device 102 over the network 104.

The second control circuit 1634 can be implemented in a number of different manners. For example, the second control circuit 1634 can be a processor, an embedded processor, a microprocessor, hardware control logic, a hardware finite state machine (FSM), a digital signal processor (DSP), or a combination thereof.

The second control circuit 1634 can include a second control interface 1644. The second control interface 1644 can be used for communication between the second control circuit 1634 and other functional units or circuits in the second device 106. The second control interface 1644 can also be used for communication that is external to the second device 106.

The second control interface 1644 can receive information from the other functional units/circuits or from external sources or can transmit information to the other functional units/circuits or to external destinations. The external sources and the external destinations refer to sources and destinations external to the second device 106.

The second control interface 1644 can be implemented in different ways and can include different implementations depending on which functional units/circuits or external units/circuits are being interfaced with the second control interface 1644. For example, the second control interface 1644 can be implemented with a pressure sensor, an inertial sensor, a microelectromechanical system (MEMS), optical circuitry, waveguides, wireless circuitry, wireline circuitry, or a combination thereof.

The second storage circuit 1646 can store the second software 1642. The second storage circuit 1646 can also store the information such as data representing incoming patient image 114, data representing previously presented image, sound files, or a combination thereof. The second storage circuit 1646 can be sized to provide the additional storage capacity to supplement the first storage circuit 1614.

For illustrative purposes, the second storage circuit 1646 is shown as a single element, although it is understood that the second storage circuit 1646 can be a distribution of storage elements. Also, for illustrative purposes, the compute system 100 is shown with the second storage circuit 1646 as a single hierarchy storage system, although it is understood that the compute system 100 can include the second storage circuit 1646 in a different configuration. For example, the second storage circuit 1646 can be formed with different storage technologies forming a memory hierarchal system including different levels of caching, main memory, rotating media, or off-line storage.

The second storage circuit 1646 can be a controller of a volatile memory, a nonvolatile memory, an internal memory, an external memory, or a combination thereof. For example, the second storage circuit 1646 can be a controller of a nonvolatile storage such as non-volatile random-access memory (NVRAM), Flash memory, disk storage, or a volatile storage such as static random access memory (SRAM).

The second storage interface 1648 can receive information from the other functional units/circuits or from external sources or can transmit information to the other functional units/circuits or to external destinations. The external sources and the external destinations refer to sources and destinations external to the second device 106.

The second storage interface 1648 can include different implementations depending on which functional units/circuits or external units/circuits are being interfaced with the second storage circuit 1646. The second storage interface 1648 can be implemented with technologies and techniques similar to the implementation of the second control interface 1644.

The second communication circuit 1636 can enable external communication to and from the second device 106. For example, the second communication circuit 1636 can permit the second device 106 to communicate with the first device 102 over the network 104.

The second communication circuit 1636 can also function as a communication hub allowing the second device 106 to function as part of the network 104 and not limited to be an endpoint or terminal unit or circuit to the network 104. The second communication circuit 16 36 can include active and passive components, such as microelectronics or an antenna, for interaction with the network 104.

The second communication circuit 1636 can include a second communication interface 1650. The second communication interface 1650 can be used for communication between the second communication circuit 1636 and other functional units or circuits in the second device 106. The second communication interface 1650 can receive information from the other functional units/circuits or can transmit information to the other functional units or circuits.

The second communication interface 1650 can include different implementations depending on which functional units or circuits are being interfaced with the second communication circuit 1636. The second communication interface 1650 can be implemented with technologies and techniques similar to the implementation of the second control interface 1644.

The second communication circuit 1636 can couple with the network 104 to send information to the first device 102. The first device 102 can receive information in the first communication circuit 1616 from the second device transmission 1610 of the network 104. The compute system 100 can be executed by the first control circuit 1612, the second control circuit 1634, or a combination thereof. For illustrative purposes, the second device 106 is shown with the partition containing the second user interface 1638, the second storage circuit 1646, the second control circuit 1634, and the second communication circuit 1636, although it is understood that the second device 106 can include a different partition. For example, the second software 1642 can be partitioned differently such that some or all of its function can be in the second control circuit 1634 and the second communication circuit 1636. Also, the second device 106 can include other functional units or circuits not shown in FIG. 5 for clarity.

The functional units or circuits in the first device 102 can work individually and independently of the other functional units or circuits. The first device 102 can work individually and independently from the second device 106 and the network 104.

The functional units or circuits in the second device 106 can work individually and independently of the other functional units or circuits. The second device 106 can work individually and independently from the first device 102 and the network 104.

The functional units or circuits described above can be implemented in hardware. For example, one or more of the functional units or circuits can be implemented using a gate array, an application specific integrated circuit (ASIC), circuitry, a processor, a computer, integrated circuit, integrated circuit cores, a pressure sensor, an inertial sensor, a microelectromechanical system (MEMS), a passive device, a physical non-transitory memory medium containing instructions for performing the software function of the detection mechanism 116, a portion therein, or a combination thereof.

For illustrative purposes, the compute system 100 is described by operation of the first device 102 and the second device 106. It is understood that the first device 102 and the second device 106 can operate any of the modules and functions of the compute system 100 including a distribution of the functions of the detection mechanism 116.

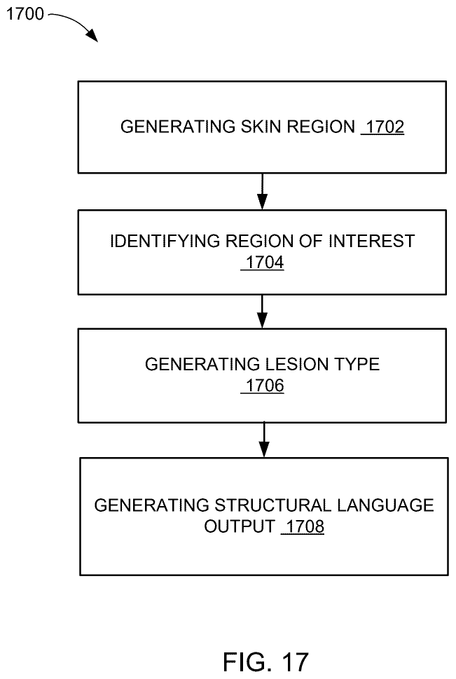

Referring now to FIG. 17, therein is shown is a flow chart of a method 1700 of operation of a compute system 100 of FIG. 1 in an embodiment of the present invention. The method 1700 includes: generating a skin region based on a patient image in a block 1702; identifying a region of interest from the skin region in a block 1704; generating a lesion type based on the region of interest in a block 1706; and generating a structural language output based on a category related to the lesion type for displaying on a device to assist in diagnosis in a block 1708.

The resulting method, process, apparatus, device, product, and/or system is straightforward, cost-effective, uncomplicated, highly versatile, accurate, sensitive, and effective, and can be implemented by adapting known components for ready, efficient, and economical manufacturing, application, and utilization. Another important aspect of an embodiment of the present invention is that it valuably supports and services the historical trend of reducing costs, simplifying systems, and increasing performance.

These and other valuable aspects of an embodiment of the present invention consequently further the state of the technology to at least the next level.

While the invention has been described in conjunction with a specific best mode, it is to be understood that many alternatives, modifications, and variations will be apparent to those skilled in the art in light of the foregoing description. Accordingly, it is intended to embrace all such alternatives, modifications, and variations that fall within the scope of the included claims. All matters set forth herein or shown in the accompanying drawings are to be interpreted in an illustrative and non-limiting sense.

1. A method of operation of a compute system comprising:
generating a skin region based on a patient image;
identifying a region of interest from the skin region;
generating a lesion type based on the region of interest;
generating a bounding box around the region of interest;
calculating a feature from pixel data inside the region of interest bounded by the bounding box; and
generating a structural language output based on a category related to the lesion type and the feature corresponding to the category for displaying on a device to assist in diagnosis.

2. The method as claimed in claim 1 further comprising generating a questionnaire based on the lesion type to distinguish between different diseases.

3. The method as claimed in claim 1 further comprising generating a possible disease class for the lesion type for displaying for review.

4. The method as claimed in claim 1 wherein generating the structural language output includes generating the structural language output based on a body part, the skin region, the lesion type, or a combination thereof.

5. The method as claimed in claim 1 wherein:
generating the structural language output includes generating the structural language output with a possible disease class for the bounding box.

6. (canceled)

7. The method as claimed in claim 1 wherein generating the structural language output includes generating the structural language output with the category including a number of lesions.

8. A compute system comprising:
a control circuit, including a processor, configured to:
generate a skin region based on a patient image;
identify a region of interest from the skin region;
generate a lesion type based on the region of interest;
generate a bounding box around the region of interest;
calculate a feature from pixel data inside the region of interest bounded by the bounding box; and
generate a structural language output based on a category related to the lesion type and the feature corresponding to the category for displaying on a device to assist in diagnosis.

9. The compute system as claimed in claim 8 wherein the control circuit is further configured to generate a questionnaire based on the lesion type to distinguish between different diseases.

10. The compute system as claimed in claim 8 wherein the control circuit is further configured to generate a possible disease class for the lesion type for displaying for review.

11. The compute system as claimed in claim 8 wherein the control circuit is further configured to generate the structural language output based on a body part, the skin region, the lesion type, or a combination thereof.

12. The compute system as claimed in claim 8 wherein the control circuit is further configured to:
generate the structural language output with a possible disease class for the bounding box.

13. (canceled)

14. The compute system as claimed in claim 8 wherein the control circuit is further configured to generate the structural language output with the category including a number of lesions.

15. A non-transitory computer-readable medium storing an instruction that, when executed by a control circuit of a compute system, causes the control circuit to perform functions comprising:
generating a skin region based on a patient image;
identifying a region of interest from the skin region;
generating a lesion type based on the region of interest;
generating a bounding box around the region of interest;
calculating a feature from pixel data inside the region of interest bounded by the bounding box; and
generating a structural language output based on a category related to the lesion type and the feature corresponding to the category for displaying on a device to assist in diagnosis.

16. The non-transitory computer-readable medium as claimed in claim 15 further comprising generating a questionnaire based on the lesion type to distinguish between different diseases.

17. The non-transitory computer-readable medium as claimed in claim 15 further comprising generating a possible disease class for the lesion type for displaying for review.

18. The non-transitory computer-readable medium as claimed in claim 15 wherein generating the structural language output includes generating the structural language output based on a body part, the skin region, the lesion type, or a combination thereof.

19. The non-transitory computer-readable medium as claimed in claim 15 wherein:
generating the structural language output includes generating the structural language output with a possible disease class for the bounding box.

20. (canceled)

21. The method as claimed in claim 1 wherein:
generating the structural language output includes generating the structural language output with a possible disease class; and
further comprising:
updating the structural language output based on a clinician input indicating a non-concurrence with the possible disease class presented in the structural language output.

22. The compute system as claimed in claim 8 wherein the control circuit is further configured to:
generate the structural language output with a possible disease class; and
update the structural language output based on a clinician input indicating a non-concurrence with the possible disease class presented in the structural language output.

23. The non-transitory computer-readable medium as claimed in claim 15 wherein:
generating the structural language output includes generating the structural language output with a possible disease class; and
further comprising:
updating the structural language output based on a clinician input indicating a non-concurrence with the possible disease class presented in the structural language output.
